(19) United States
(12) Patent Application Publication
(10) Pub. No.: US 2026/0130696 A1
(43) Pub. Date: May 14, 2026

(54) Systems, Devices, and Methods for Transpedicular Spinal Treatment

(71) Applicant: Fortissimo Spine Corporation, Memphis, TN (US)

(72) Inventors: Andrew M. Dickson, Knoxville, TN (US); Kevin T. Foley, Germantown, TN (US)

(21) Appl. No.: 19/388,735

(22) Filed: Nov. 13, 2025

Related U.S. Application Data
(60) Provisional application No. 63/720,098, filed on Nov. 13, 2024.

(51) Int. Cl. A61B 17/70; A61B 17/34; A61B 90/00

(52) U.S. Cl. CPC A61B 17/7061; A61B 17/3423; A61B 17/3472; A61B 90/39; A61B 2090/3966

(57) ABSTRACT

A transpedicular spinal treatment system may include a transpedicular access port having an aperture positioned proximally to the distal end of the transpedicular access port. The transpedicular access port may be configured to enter a vertebra through the pedicle bone. A flexible member may be configured to translate along the lengthwise axis of the transpedicular access port. The aperture may allow the flexible member to extend from the aperture in a direction at least partially oblique to the lengthwise axis of the transpedicular access port. The flexible member may have an opening proximal to the distal end of the flexible member.

### CROSS-REFERENCE TO RELATED APPLICATIONS

The present application claims priority to and the benefit of U.S. Provisional Ser. No. 63/720,098 , filed on Nov. 13, 2024, and entitled “SYSTEMS, DEVICES, AND METHODS FOR TRANSPEDICULAR SPINAL TREATMENT” which is hereby incorporated herein by reference in its entirety.

### TECHNICAL FIELD

The present disclosure relates generally to orthopedic treatment, medicine, and/or surgery and more specifically to spinal stabilization surgery.

### BACKGROUND

Some patients may experience back pain and/or discomfort due to deterioration and/or weakening of the bone within one or more vertebrae. This pain or discomfort may be medically treated by inserting material into the vertebra to stabilize fractures within the bone and/or reduce the load applied on specific areas of the bone. Known techniques involve forming a cavity within the vertebra and filling the cavity with bone cement. Such techniques may be ineffective at controllably accessing, filling, and/or supporting the vertebra which may not alleviate patient pain and discomfort, or such techniques may not withstand patient movements over time. Thus, there remains a need for effective ways to stabilize a vertebra.

### SUMMARY

Embodiments of the present disclosure include systems, devices, and methods of stabilizing a vertebra.

In an exemplary aspect, a transpedicular spinal treatment system is provided that includes a vertebral body access port having an aperture positioned proximally to the distal end of the vertebral body access port, the vertebral body access port configured to enter a vertebra through a pedicle bone. The system further includes a flexible member configured to translate along the lengthwise axis of the vertebral body access port, the aperture allowing the flexible member to extend from the aperture in a direction at least partially oblique to the lengthwise axis of the vertebral body access port, the flexible member having an opening proximal to the distal end of the flexible member.

In another exemplary aspect, a transpedicular spinal treatment system is provided that includes a transpedicular access port having a thread and an aperture positioned proximal to the distal end of the transpedicular access port, the aperture extending longitudinally across at least two threads of the transpedicular access port, the transpedicular access port configured to enter a vertebra through a pedicle bone. The system further includes a flexible member including an internal lumen and configured to translate along the lengthwise axis of the transpedicular access port, the aperture allowing the flexible member to extend from the aperture in a direction at least partially oblique to the lengthwise axis of the transpedicular access port. The system also includes a stent positioned proximally to the distal end of the flexible member and a balloon catheter configured to move along the internal lumen and configured to expand and abut against the stent causing the stent to expand into marrow of the vertebra.

In still a further aspect, a transpedicular spinal treatment system is provided comprising a transpedicular access port having an aperture positioned proximally to the distal end of the transpedicular access port, the transpedicular access port configured to enter a vertebra through a pedicle bone. The system further includes a calibration device configured to adjust the orientation of the transpedicular access port and the aperture relative to the vertebra along with a flexible member configured to translate along the lengthwise axis of the transpedicular access port. The aperture allowing the flexible member to extend from the aperture in a direction at least partially oblique to the lengthwise axis of the transpedicular access port, the flexible member having an opening proximal to the distal end of the flexible member, and the flexible member configured to deliver bone void filler to the interior of the vertebra.

In still a further embodiment, a vertebral body access port is provided that comprises a radiopaque tubular body having a central longitudinal axis and a sidewall extending along said axis, the body including a distal portion and a proximal portion interconnected by an central portion having an outer diameter and defining an inner working lumen having a first inner diameter. The distal portion having a distal tip and defining an access window opening extending through the sidewall. The working lumen terminating into a deflection surface formed in the distal portion adjacent the access window. In some embodiments, the distal portion further including an orientation indicator detectable via external imaging systems configured to provide an indication of the axial orientation of the access window about the longitudinal axis. In further aspects, the central portion includes a bone engaging structure on the sidewall to inhibit axial movement of the body within the bone along the longitudinal axis. In another aspect, the sidewall is closed along the longitudinal length. The proximal portion having an enlarged head defining a tool engagement surface for connecting with an insertion tool and an internal passage communicating with the inner working lumen. In one alternative, the internal passage has a dimension that is equal to or larger than the first inner diameter and the tool engagement surface having a dimension that is larger than the first inner diameter. The tool engagement surface further includes an alignment feature configured to permit engagement of the insertion tool in a predefined axial orientation relative to the tubular body.

Additional aspects, features, and advantages of the present disclosure will become apparent from the following detailed description.

### BRIEF DESCRIPTION OF THE DRAWINGS

The accompanying drawing figures incorporated in and forming a part of this specification illustrate several aspects of the disclosure, and together with the description, serve to explain the principles of the disclosure.

FIG. 1 illustrates a side view of an example of a patient and a spinal treatment system, according to some aspects of the present disclosure.

FIG. 2 illustrates a side perspective view of an example of at least a portion of a spine, according to some aspects of the present disclosure.

FIG. 3 illustrates a top cross-sectional view of an example of a spinal treatment system and a vertebra, according to some aspects of the present disclosure.

FIG. 4 illustrates a top cross-sectional view of an example of a spinal treatment system and a vertebra, according to some aspects of the present disclosure.

FIG. 5 illustrates a top cross-sectional view of an example of a spinal treatment system and a vertebra, according to some aspects of the present disclosure.

FIG. 6 illustrates a top cross-sectional view of an example of a spinal treatment system and a vertebra, according to some aspects of the present disclosure.

FIG. 7 illustrates a top cross-sectional view of an example of a spinal treatment system and a vertebra, according to some aspects of the present disclosure.

FIG. 8 illustrates a top cross-sectional view of an example of a spinal treatment system and a vertebra, according to some aspects of the present disclosure.

FIG. 9 illustrates a cross-sectional view of an example of a cutter, according to some aspects of the present disclosure.

FIG. 10 illustrates a cross-sectional view of an example of a cutter, according to some aspects of the present disclosure.

FIG. 11 illustrates a top cross-sectional view of an example of a spinal treatment system and a vertebra, according to some aspects of the present disclosure.

FIG. 12 illustrates a partial top cross-sectional view of an example of a spinal treatment system and a vertebra, according to some aspects of the present disclosure.

FIG. 13 illustrates a partial top cross-sectional view of an example of a spinal treatment system and a vertebra, according to some aspects of the present disclosure.

FIG. 14 illustrates a further embodiment of a spinal treatment system and vertebra, according to another aspect of the present disclosure.

FIG. 15 illustrates an example of a method of stabilizing a vertebra, according to some aspects of the present disclosure.

FIG. 16 illustrates a side perspective view of an example of a spinal treatment system, according to some aspects of the present disclosure.

FIG. 17 illustrates a side view of an example of a spinal treatment system, according to some aspects of the present disclosure.

FIG. 18 illustrates section view 18 of the example of a spinal treatment system in FIG. 16, according to some aspects of the present disclosure.

FIG. 19 illustrates section view 19 of the example of a spinal treatment system in FIG. 18, according to some aspects of the present disclosure.

FIG. 20 illustrates a partial side perspective view of an example of an awl, according to some aspects of the present disclosure.

FIG. 21 illustrates a partial side view of an example of an awl, according to some aspects of the present disclosure.

FIG. 22 illustrates a partial side view of an example of an awl, according to some aspects of the present disclosure.

FIG. 23 illustrates section view 23 of the example of an awl in FIG. 21, according to some aspects of the present disclosure.

FIG. 24 illustrates section view 24 of the example of a spinal treatment system in FIG. 16, according to some aspects of the present disclosure.

FIG. 25 illustrates a side view of an example of a spinal treatment system, according to some aspects of the present disclosure.

FIG. 26 illustrates a partial side cross-sectional view about line 26 of the example of a spinal treatment system in FIG. 25, according to some aspects of the present disclosure.

FIG. 27 illustrates a side view of an example of a spinal treatment system, according to some aspects of the present disclosure.

FIG. 28 illustrates a side cross-sectional view about line 28 of the example of a spinal treatment system in FIG. 27, according to some aspects of the present disclosure.

FIG. 29 illustrates section view 29 of the side cross-sectional view of the example of a spinal treatment system in FIG. 28, according to another aspect of the present disclosure.

FIG. 30 illustrates section view 30 of the side cross-sectional view of the example of a spinal treatment system in FIG. 28, according to some aspects of the present disclosure.

FIG. 31 illustrates the side cross-sectional view of the example of a spinal treatment system in FIG. 28 including an example of an injection tube, according to some aspects of the present disclosure.

FIG. 32 illustrates section view 32 of the side cross-sectional view of the example of a spinal treatment system in FIG. 31, according to some aspects of the present disclosure.

FIG. 33 illustrates a partial side cross-sectional view of an example of a spinal treatment system, according to some aspects of the present disclosure.

FIG. 34 illustrates section view 34 of the partial side cross-sectional view of the example of a spinal treatment system in FIG. 29, according to some aspects of the present disclosure.

FIG. 35 illustrates a partial side cross-sectional view of an example of a spinal treatment system, according to some aspects of the present disclosure.

FIG. 36 illustrates a partial top view of an example of a spinal treatment system, according to some aspects of the present disclosure.

FIG. 37 illustrates a partial side cross-sectional view of an example of a spinal treatment system, according to some aspects of the present disclosure.

FIG. 38 illustrates a partial side cross-sectional view of an example of an awl, according to some aspects of the present disclosure.

FIG. 39 illustrates a partial top view of an example of an awl, according to some aspects of the present disclosure.

FIG. 40 illustrates an example of a method of stabilizing a vertebra, according to some aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Disclosed herein are systems, devices, and methods for stabilizing a vertebra. A deliver pin, which can be in the form of a transpedicular access port, may be inserted through the pedicle and have an aperture disposed within the cancellous region of the vertebral body. A flexible awl may be directed through the aperture at an orientation nonparallel to the lengthwise axis of a transpedicular access port. In one form, the awl includes a bone tamp such as a balloon or deformable structure, that can mobilize cancellous bone to create or enlarge a void. In another aspect, the awl may support and/or deliver bone void filler materials in areas of the cancellous bone within the vertebra which are spaced a distance apart from the axes running through the pedicles of the vertebra, thereby more effectively treating spinal pain and discomfort of a patient. Once the bone void filler, such as bone cement or bioresorbable bone adhesive, is delivered into the vertebral body, the awl and deliver pin are interconnected to each other while the bone void filler hardens. After hardening, the bone void filler forms a relatively rigid construct with the delivery system such that the bone void filler may transmit vertical forces within the vertebral body while the delivery system is firmly positioned in the pedicle to resist rotational and translational forces applied to the construct. In this manner, the construct stabilizes the vertebral body to inhibit motion of the vertebral body with cured filler relative to the pedicles.

The present disclosure discusses a spinal treatment system with three-dimensional calibration capability. In some implementations, a jig, which may also be referred to as a calibration system, may assist the surgeon in precisely placing the bone access port aperture and/or steering a flexible awl within the internal marrow of the vertebra. Once the surgeon has properly positioned the awl, the awl may itself support the vertebra and/or may deliver bone void filler to the surrounding area.

In some implementations, the surgeon may use the jig in unilateral and bilateral applications. In unilateral applications, a delivery system and bone filler material are inserted through one pedicle of the vertebra. In bilateral applications, a first delivery system is placed through a first pedicle, and a second delivery system is placed through the opposite pedicle. In a bilateral procedure, the awls may have a trajectory that avoids intersection by either passing above and below each other, or by having the first awl extend more anteriorly while the second awl is posterior of the first awl. In either application, the surgeon may use the jig to adjustably steer the awl on one side of the spinal treatment system with respect to the other, thus optimally covering the internal portions of the vertebra. In some bilateral applications, the surgeon may steer the first awl and the second awl to converge at or near a point within the vertebral body. The convergence of bone void filler from the first awl and the second awl may allow the surgeon to create a connected column and/or bridge of bone void filler for greater stability as compared to two unconnected volumes created without directional control. In both unilateral and bilateral applications, the jig allows the surgeon to adjust an awl relative to at least both the transverse plane and coronal plane. In some implementations, the spinal treatment system may further include a radiographic and/or fluoroscopic imaging system which may provide the surgeon with an internal view of the awl so the surgeon may more effectively position the jig and/or steer the awl within the vertebra.

In some implementations, the surgeon may be able to precisely steer and adjust the position and/or orientation of the awl by using a specially designed cannulated transpedicular access port. The transpedicular access port may include a deflection surface at the distal end of the cannula which may deflect the awl in a direction nonparallel to the lengthwise axis of the transpedicular access port when the awl is inserted into the cannula. In some implementations, the transpedicular access port may include an aperture adjacent to the deflection surface and facing substantially perpendicularly to the lengthwise axis of the access port which may allow the awl to exit the cannula into the bone marrow of the vertebra in a direction nonparallel to the lengthwise axis of the transpedicular access port. The surgeon may use the imaging system, the jig, and/or special markings on the transpedicular access port, driver handle, and/or drill to precisely adjust the position and/or orientation of the aperture to steer and/or control the direction of extension of the awl.

In some implementations, once the awl is properly positioned, the surgeon may use the awl to apply bone void filler to the areas surrounding the awl. In some implementations, once the awl is properly positioned, the awl may include an expandable stent which may expand to support the surrounding portion of the bone and/or create a cavity to more effectively apply bone void filler.

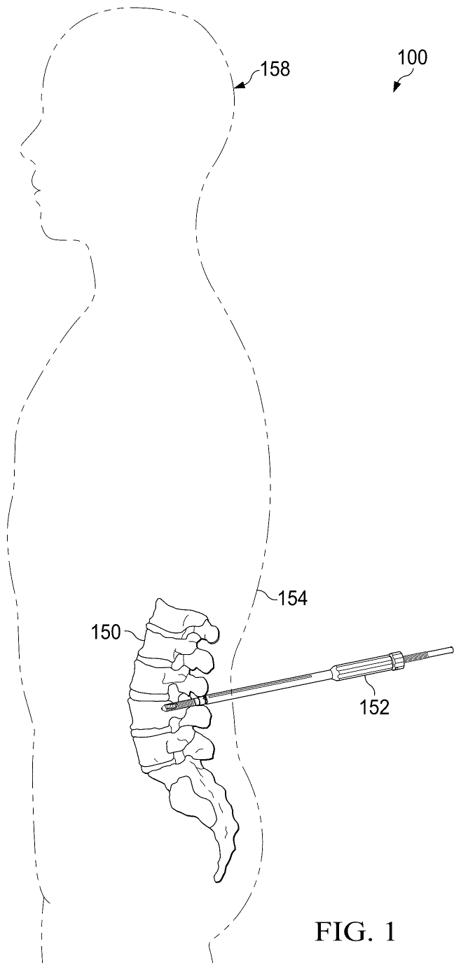

FIG. 1 illustrates a side view 100 of an example of a patient 158 and a spinal treatment system 152, according to some aspects of the present disclosure. In some implementations, the spinal treatment system 152, which may also be referred to as a transpedicular stabilization device, may enter a patient's spine 150 through the patient's posterior 154. However, while the system is shown as a transpedicular delivery system, it is contemplated that other approaches to the vertebral body may be utilized and that the system need not be limited to a transpedicular approach.

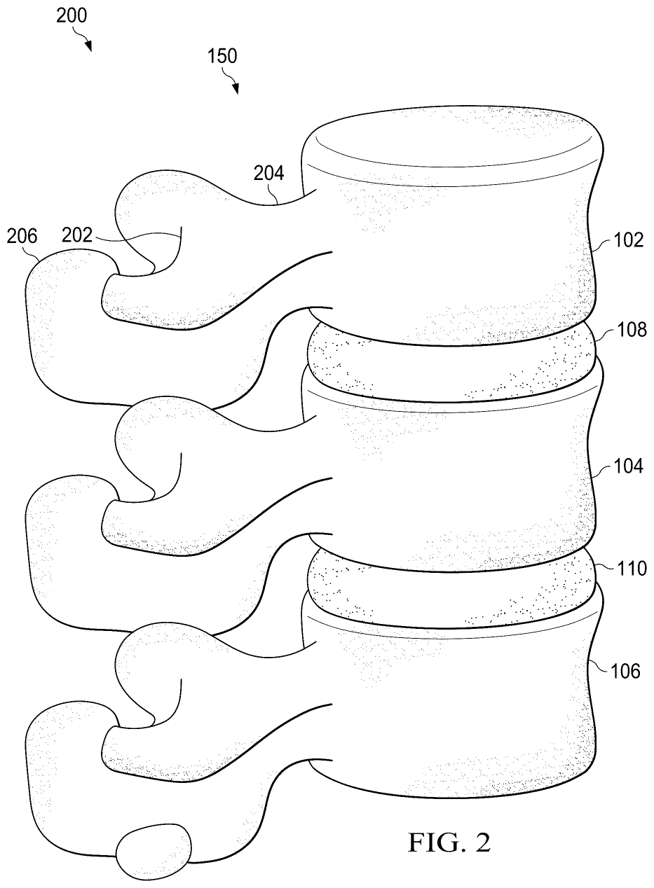

FIG. 2 illustrates a side perspective view of an example of at least a portion of a spine 150, according to some aspects of the present disclosure. FIG. 2 is an example illustration of a section 200 of the patient's spine 150. The patient's spine 150 may include a plurality of vertebrae (such as vertebrae 102, 104, 106) and a plurality of intervertebral discs (such as intervertebral discs 108, 110). A vertebra (such as vertebra 102) may include a facet joint 206. Facet joint 206 may include a transverse process 204 and a pedicle 202.

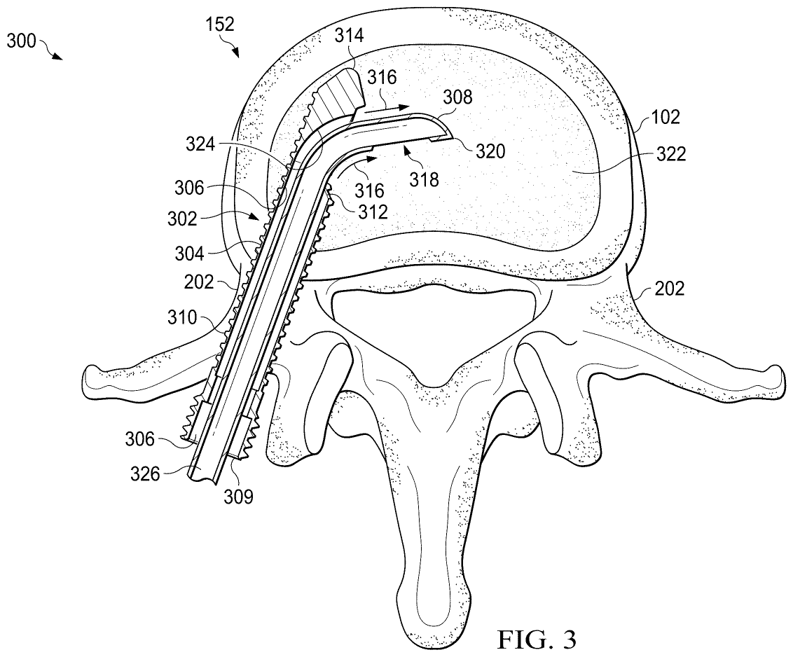

FIGS. 3-5 may illustrate examples of stages of spinal stabilization procedure. FIG. 3 illustrates a top cross-sectional view 300 of an example of a spinal treatment system 152 and a vertebra 102, according to some aspects of the present disclosure. It should be understood that the spinal treatment system 152 may include any of the features of any of the spinal treatment systems discussed herein including, for example, the spinal treatment system 1600 and/or the spinal treatment system 2500. The spinal treatment system 152 may include a transpedicular access port system 302, which may also be referred to as a bone void filler delivery device or delivery pin. In some implementations, the transpedicular access port system 302 may include at least some or all of a transpedicular access port 304 and an awl 308. The transpedicular access port 304 may also be referred to as an injection pin, an access port, a pin, an anchor, and/or any other suitable term.

In some implementations, the transpedicular access port 304 may include a cannula 306, a proximal end 309, threads 310, an aperture 312, a tip 314, and a deflection surface 324. In some implementations, the cannula 306 may be positioned in the center of the transpedicular access port 304 such that a cross-section of the cannula 306 may have a circular shape at least partially concentric with a cross-section of the transpedicular access port 304. In some implementations, the cannula 306 may extend from the proximal end 309 to the aperture 312. The cannula 306 may form a channel from an opening at the proximal end 309 to the aperture 312. In some implementations, the cannula 306 may extend from the proximal end 309 to or substantially to the tip 314. The proximal end 309 may be positioned at the end of the transpedicular access port 304 opposite the tip 314.

In some implementations, the threads 310 may extend from the proximal end 309 to the distal end of the aperture 312. In some implementations, the threads 310 may extend from the proximal end 309 to the center or proximal end of the aperture 312. In some implementations, the threads 310 may extend from the proximal end 309 to the tip 314. In some implementations, the threads 310 may extend from a distance apart from the proximal end 309 to the distal end of the aperture 312. In some implementations, the threads 310 may extend from a distance apart from the proximal end 309 to the center or proximal end of the aperture 312. In some implementations, the threads 310 may extend from a distance apart from the proximal end 309 to the tip 314. In some implementations, the threads 310 may extend from the proximal end of the aperture 312 to the tip 314. In some implementations, instead of being a threaded access port, the transpedicular access port 304 may be an injection pin with an at least partially nonthreaded exterior surface. Thus, it should be understood that depending on the implementation, the transpedicular access port 304 may be inserted and/or secured in place using any mechanism, mechanisms, method, and/or methods including, for example, threading, friction fit, compression fit, adhesive, fasteners, and/or any other suitable mechanism and/or method.

The aperture 312 may be positioned at or near the distal end of the transpedicular access port 304. In some implementations, the aperture 312 may be positioned a distance apart from the tip 314. In some implementations, the aperture 312 may be positioned adjacent or substantially adjacent to the tip 314. The aperture 312 may be at least sufficient size and shape to allow the awl 308 to exit the cannula 306 through the aperture 312. It should be understood that the length of the aperture 312 relative to the lengthwise axis of the transpedicular access port 304 may be any length including, for example, about ⅛ inches to ½ inches.

The tip 314 may be positioned at the distal end of the transpedicular access port 304. In some implementations, the tip 314 may be pointed or include a self-tapping feature which may allow the transpedicular access port 304 to more easily pierce the bone 102. In some implementations, the diameter of the transpedicular access port 304 may narrow gradually towards the tip 314.

In some implementations, a deflection surface 324 may be positioned at the distal end of the cannula 306 and/or adjacent to the distal end of the aperture 312. The deflection surface 324 may be configured to deflect the awl 308 from translating fully or substantially to the lengthwise axis of the transpedicular access port along the cannula 306 to exiting the aperture 312. In some implementations, the deflection surface 324 may be oblique to the lengthwise axis of the transpedicular access port 304. In some implementations, the deflection surface may be curved and/or may include a fillet or chamfer which may extend from a side of the cannula 306 to the distal end of the aperture 312. In some implementations, at least part of the deflection surface 324 may be fully or substantially perpendicular to the lengthwise axis of the transpedicular access port 304. In some implementations, the deflection surface 324 may cause the awl 308 to transition from, being fully or substantially parallel to the lengthwise axis of the transpedicular access port 304 to being nonparallel and/or having at least one perpendicular vector component relative to the lengthwise axis of the transpedicular access port 304. In some implementations, the deflection surface 324 may force the awl 308 in direction 316.

The awl 308 may include a window 318, a point 320, and a lumen 326. In some implementations, the awl 308 may be at least partially made of a flexible material or materials. It should be understood that depending in the embodiment, the awl 308 may be made of any material or materials including, for example, flexible polymer material, polyether ether ketone (PEEK), thermoplastic elastomer, super-elastic metallic material, nickel titanium alloy (nitinol), plastic, rubber, silicone, foam, urethane, polyester, polyvinyl chloride, polyethylene, polystyrene, polycarbonate, polypropylene, polymethyl, nylon, acrylonitrile butadiene styrene, polylactic acid (PLA), polyglycolic acid (PGA), polylactic-co-glycolic acid (PLGA), polydioxanone (PDO), polycaprolactone (PCL), magnesium, collagen, hyaluronic acid, alginic acid, gelatin, chitin, chitosan, carboxymethylcellulose (CMC), glycosaminoglycan, fibrinogen, tricalcium phosphate, carbonate apatite, any biodegradable material, and/or any other suitable material. In some implementations, the awl 308 may be at least partially constructed from laser-cut, machined, and/or 3D-printed material(s) and/or tubing. In some implementations, the distal end of the awl 308 may be more flexible and/or may be made of a more flexible material than the rest of the awl 308. It should be understood that the awl 308 may have any length including, for example, a length based on the size of the targeted area with the bone 102, a length of about 2 inches to 20 inches, and/or any other length.

The window 318 may be positioned at or near the distal end of the awl 308. In some implementations, the window 318 may be positioned a distance apart from the point 320. In some implementations, window 318 may be the same or substantially the same distance away from the point 320 and the aperture 312 from the tip 314. In some implementations, the window 318 may be positioned adjacent or substantially adjacent to the point 320. In some implementations, the window 318 may have the same or substantially the same length and/or width as the aperture 312. However, it should be understood that the length of the window 318 relative to the lengthwise axis of the awl 308 may be any length including, for example, about ⅛ inches to ½ inches.

The point 320 may be positioned at the distal end of the awl 308. In some implementations, the point 320 may allow the awl 308 to more easily pierce the bone 102. In some implementations, the diameter of the awl 308 may narrow gradually towards the point 320 on one or two sides. In some implementations, the shape of the awl 308 adjacent to the point 320 may match or substantially align with the shape of the deflection surface 324 which may allow the deflection surface 324 to more effectively deflect the awl 308 out of the aperture 312.

In some implementations, the lumen 326 may be positioned in the center of the awl 308 such that a cross-section of the lumen 326 may have a circular shape at least partially concentric with a cross-section of the awl 308. In some implementations, the lumen 326 may extend the entire length of the awl 308. In some implementations, the lumen 326 may extend from the proximal end of the awl 308 to the window 318. The lumen 326 may form a channel from an opening at the proximal end of the awl 308 to the window 318. In some implementations, the lumen 326 may extend from the proximal end of the awl 308 to or substantially to the point 320. The lumen 326 may provide a fluid connection between the proximal end of the awl 308 and the window 318.

In some implementations, the transpedicular access port system 302 may be used as part of a surgical procedure to stabilize one or more vertebrae 102 within a patient's spine 150. In some implementations, a surgeon may insert one or more Kirschner wires (K wires) (not shown) into a hole in the bone 102 extending through the pedicle 202 and into the marrow 322. In some implementations, the cannula 306 may be positioned about one of the one or more K wires which may allow the surgeon to align the position and/or orientation of the transpedicular access port 304 using the K wire and/or then drill the transpedicular access port 304 into the hole in the bone 102. In some implementations, the transpedicular access port 304 may be inserted and/or drilled directly into the insertion location (e.g., the pedicle) and/or hole in the bone after bone preparation. Thus, in some implementations, one or more K wires may not be utilized to position and/or orient the transpedicular access port 304. It should be understood that the transpedicular access port 304 may be drilled any angle and distance into the bone including, for example, about one inch to three inches. Thus, when the transpedicular access port is positioned within the bone 102, the proximal end 309 may be external to the bone 102, the shaft of the transpedicular access port 304 may be at least partially positioned within the pedicle 202 and/or the marrow 322, and/or the tip 314 may extend into the marrow 322. In some implementations, the surgeon may use orientations marking on the transpedicular access port 304 and/or the driver handle (not shown) to orient the aperture 312 relative to the bone 102. In some implementations, the surgeon may use x-ray and/or fluoroscopy to view the aperture 312 relative to the bone 102 and/or orient the aperture 312 relative to the bone 102. For example, in some implementations, the surgeon may prefer to position the aperture 312 to at least partially face fully or substantially perpendicularly to the lengthwise axis of the transpedicular access port 304. In some implementations, the surgeon may prefer to position the aperture 312 to face inwardly toward the portion of the bone between the pedicles 202. In some implementations, the surgeon may prefer to position the aperture 312 to face medially.

In some implementations, the one or more K wires may be used to position a drill (not shown) which may drill a hole for the transpedicular access port 304 fully or substantially at the location of the one or more K wires. In some implementations, the cannula 306 may extend the entire length of the transpedicular access port 304, and the transpedicular access port 304 may be positioned and/or oriented by moving the transpedicular access port 304 over one or more K wires such that the K wire is positioned at least partially within the cannula 306 and then drilling the transpedicular access port 304 into place. In some implementations, the cannula 306 may have a smaller cross-sectional diameter on the distal side of the deflection surface 324 than cross-sectional diameter on the proximal side of the deflection surface and/or a smaller diameter than the cross-sectional diameter of the awl 308 such that the awl 308 will not move into the distal side of the cannula 306 instead of the aperture 312.

Once the transpedicular access port 304 and/or aperture are positioned, the surgeon may insert the awl 308 into the cannula 306 at the proximal end 309 and lead the awl 308 through the cannula 306 until the point 320 of the awl 308 makes contact with the deflection surface 324. The flexible awl 308 moves further into the cannula 306, the deflection surface 324 may deflect distal end of the awl out of the aperture 312 and into the marrow 322. The surgeon may continue to push the awl 308 into the cannula 306 until the point 320 is a distance apart from the aperture 312. In some implementations, when the awl 308 is being inserted into the marrow 322, a push rod (not shown) may be inserted into the awl 308 to block marrow from entering the window 318. The push rod may later be removed to allow the window 318 to open. It should be understood that depending on the implementation, the push rod may be made of any material or materials including, for example, the same or different material or materials as the awl 308. In some implementations, the push rod may be made of a flexible material or materials. In some implementations, the surgeon may use x-ray and/or fluoroscopy to view the awl 308 relative to the bone 102 and/or orient awl 308 relative to the bone 102. For example, in some implementations, the surgeon may prefer to position the awl 308 to at least partially extend fully or substantially perpendicularly to the lengthwise axis of the transpedicular access port 304. In some implementations, the angle of the lengthwise axis of the extension of the awl 308 from the aperture 312 relative to the medial side of the transpedicular access port 304 may be an obtuse angle. In some implementations, the posterior-facing side of the awl 308 may at least partially form an at least partially radial arc extending from the medial side of the transpedicular access port 304 fully or substantially to or through the sagittal plane of the patient. In some implementations, the surgeon may prefer to position the awl 308 to extend inwardly toward the portion of the bone between the pedicles 202. In some implementations, the surgeon may prefer to position the awl 308 to extend medially.

Although FIG. 3 illustrates an example of one spinal treatment system 152, in some implementations there may be a second spinal treatment system (not shown) which may mirror the spinal treatment system 152 about the sagittal plane. It should be understood that depending on the implementation, this second spinal treatment system may incorporate at least some or all of any of the aspects or features of any on the examples disclosed herein including, for example, the disclosure relating to at least FIGS. 3-8. Implementations with one spinal treatment system 152 may be referred to as unilateral while implementations with two spinal treatment systems may be referred to as bilateral.

In some bilateral implementations, the bone void filler 518 may be delivered to both awls 308 simultaneously, substantially simultaneously, and/or within close proximity (e.g., within about 30 seconds). In some bilateral implementations, the transpedicular access ports 304 may be adjusted such that at least a portion of each awl 308 is positioned in a different transverse plane. For example, the second awl may be positioned cephalad or caudal (e.g., higher or lower) than the first awl 308. In some bilateral implementations, the transpedicular access ports 304 may be adjusted such that at least a portion of each awl 308 is positioned in a different plane fully or substantially parallel to the coronal plane. For example, the second awl may be positioned anterior or posterior to the first awl 308. In some bilateral implementations, the transpedicular access ports 304 may be adjusted such that at least a portion of each awl 308 is positioned in a different transverse plane and a different plane fully or substantially parallel to the coronal plane.

In some implementations, orientation and/or positioning of the second transpedicular access port 304 in some bilateral implementations may be accomplished with a jig (not shown), which may also be referred to as a calibration system, a calibration device, a steering device, and/or a control, and which may be at least partially external to the patient's body. The positioning of the second access port 304 may adjusted by using the jig which may be placed in alignment and/or on the lengthwise axis of the first transpedicular access port 304. The jig may include a connected member, such as a tube, on the other side of the sagittal plane that may direct bone preparation and/or orientation of the second transpedicular access port such that the extension of the awl 308 of the second transpedicular access port 304 is on a fully or substantially non-collision path with the awl 308 of the first transpedicular access port 304. For example, the jig may include a connected member, such as a tube, on the other side of the sagittal plane that may direct bone preparation and/or orientation of the second transpedicular access port such that the extension of the awl 308 of the second transpedicular access port 304 is cephalad or caudal and/or anterior or posterior with the awl 308 of the first transpedicular access port 304. In some implementations, the jig may be used to align and/or orient the transpedicular access ports to have the same or substantially the same mirrored position relative to the sagittal plane. In some aspects, the steering of the awl 308 may be limited to within a single vertebra and/or may be directed primarily or fully toward the center of a single vertebra.

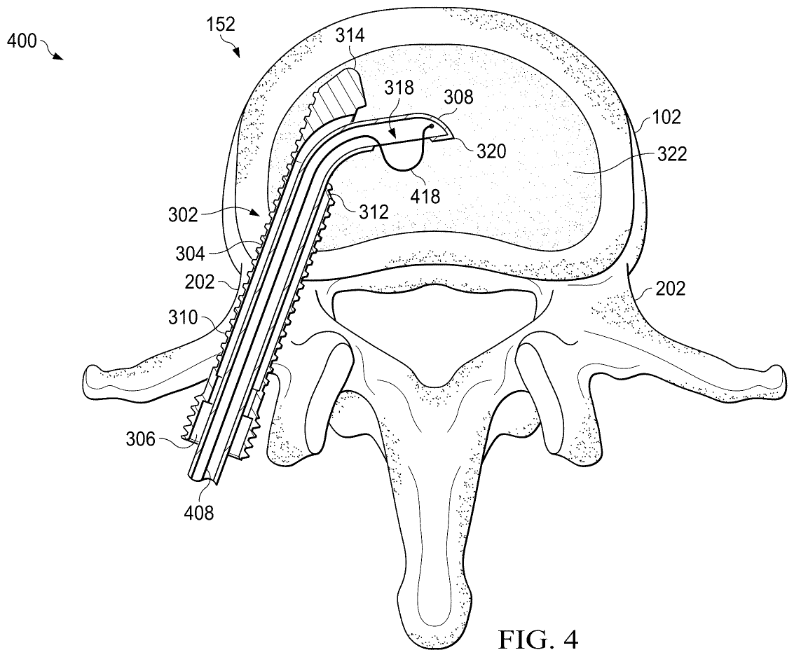

FIG. 4 illustrates a top cross-sectional view 400 of an example of a spinal treatment system 152 and a vertebra 102, according to some aspects of the present disclosure.

In some implementations, the awl 308 includes a catheter 408 connected to a deployable bone tamp 418. The bone tamp 418 may be positioned at the distal end of the catheter 408 or a distance away from the distal end of the catheter 408. In some implementations, when the bone tamp 418 reaches the window 318 it may be deployable such that the bone tamp extends into the marrow 322 and clears a cavity in the marrow 322. In some implementations, the bone tamp 418 may be deployed once the bone tamp 418 reaches the window 318 by elastic energy forcing the bone tamp 418 into the window 318 using, for example, advancement of the catheter 408 towards the distal end of the awl, an elastically compressed material, spring, fold, balloon catheter, and/or any other mechanism for creating elastic potential energy. In some implementations, the bone tamp 418 may be remotely actuatable by a user such as a surgeon. In some implementations, the bone tamp 418 may be remotely actuatable by a user such as a surgeon by using a valve to compress one or more fluids in the lumen 326, causing the pressure of the one or more fluids in the lumen 326 to force the bone tamp 418 out from the window 318. In some implementations, the bone tamp 418 may include a plate (not shown) which may push into and displace the marrow 322 adjacent and/or proximal to the window 318. The plate may be made of a relatively firm material or materials relative to the material or materials of the awl 308 including, for example, metal, alloy, steel, stainless steel, iron, titanium, and/or any other suitable material.

Once the awl 308 is positioned, for example according to any steps described with respect to FIG. 3, the surgeon may deploy the bone tamp 418 to clear bone marrow 322 adjacent and/or proximal to the window 318 which may create or expand a void and/or cavity in the marrow 322.

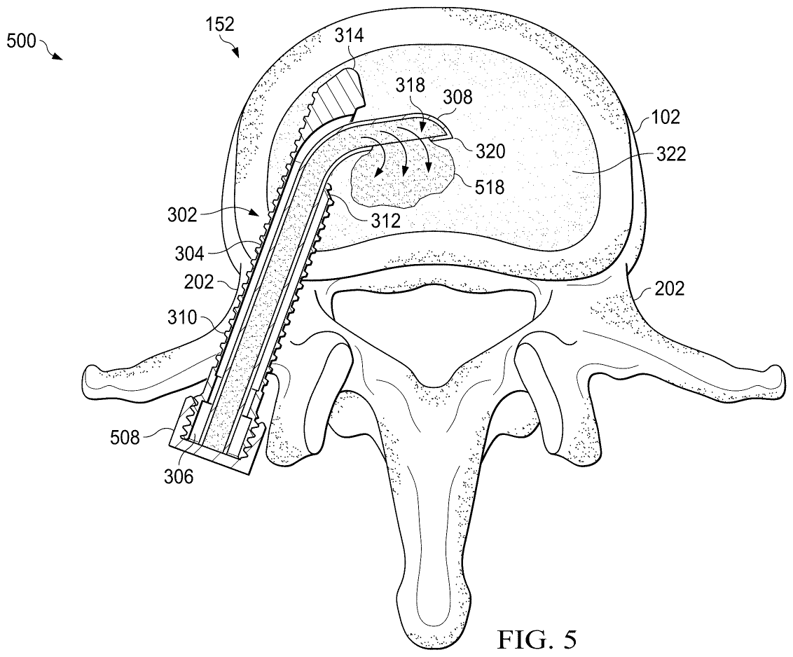

FIG. 5 illustrates a top cross-sectional view 500 of an example of a spinal treatment system 152 and a vertebra 102, according to some aspects of the present disclosure. Once the awl 308 is positioned, for example according to any steps described with respect to FIG. 3, and/or the catheter 408 is positioned according to any steps described with respect to FIG. 4, the surgeon may withdraw the bone tamp 418 from the lumen 326. In some implementations, the surgeon may then insert bone void filler 518 into the lumen 326, and the bone void filler 518 may flow from the proximal end of the lumen 326, through the lumen 326, and out of the window 318 into the marrow 322. In some implementations, the surgeon may insert a fluid delivery catheter (not shown), which may also be referred to as filler tube and/or a bone void filler delivery duct, into the lumen 326 which may dispense bone void filler 518 at or near the window 318. In some implementations, the bone tamp 418 may clear some of the marrow 322 away from the window 318 before the bone void filler 518 is dispensed. This may allow the bone void filler 518 to flow into the marrow 322 more effectively. It should be understood that the bone void filler 518 may be any type of material or materials including, for example, bone cement, bone adhesive, bioresorbable bone adhesive such as Tetranite offered by RevBio, calcium phosphate, calcium sulfate, bone marrow aspirate, bioresorbable material, biologic, bone graft, bone marrow transplant, bone growth promoting materials and/or any other suitable material. In some implementations, the combination of the medial support of the bone void filler 518 and the support of the one or more transpedicular access ports 304 along the lengthwise axis of the transpedicular access port 304 within the one or more pedicles 202 allows for the vertebra(e) to be more effectively supported as the pedicle 202 portion of the vertebra 102 is generally stronger than other parts of the vertebra 102 and/or surrounding cartilage and tissue.

In some implementations, once the bone void filler 518 has been dispensed into the bone marrow 322, the surgeon may seal the proximal end of the lumen 326 and/or cannula 306 with a cap 508. The cap 508 fixes the awl 308 within the transpedicular access port 304 and may prevent the bone void filler 518 from seeping from the proximal end of the lumen 326 and/or cannula 306 into the patient's body which could cause damage and/or discomfort to the patient's tissue. Additional implementations of the cap 508 may be discussed further herein.

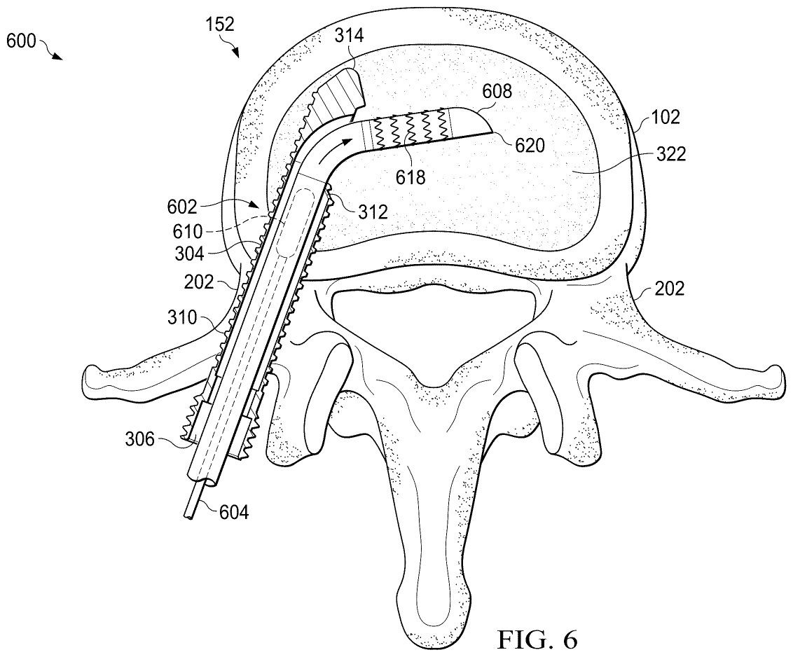

FIGS. 6-8 may illustrate examples of stages of spinal stabilization procedure. FIG. 6 illustrates a top cross-sectional view 600 of an example of a spinal treatment system 152 and a vertebra 102, according to some aspects of the present disclosure. In some implementations, a transpedicular access port system 602 may include some or all of a transpedicular access port 304, an awl 608, and a balloon catheter 604. The transpedicular access port 304 may include any of the applicable aspects or features of any of the implementations of a transpedicular access port discussed herein including, for example, any implementations discussed with respect to FIGS. 3-5.

The awl 608 may include any of the applicable aspects or features of any of the implementations of an awl discussed herein including, for example, any implementations discussed with respect to FIGS. 3-5. The awl 608 may include a stent 618. The stent may be positioned at or a distance away from the point 320. In some implementations the stent 618 may extend entirely around the outer circumference of the awl 608. In some implementations, the stent 618 may extend only partially around the outer circumference on the awl 608, for example, being positioned within the window 318. In some implementations, the stent 618 may be inset into the awl 608 such that the stent 618 has a smaller cross-sectional diameter than that of the lumen 326. In some implementations, the stent 618 may have a cross-sectional diameter larger than that of the lumen 326 but smaller than that of the cannula 306.

In some implementations, the balloon catheter 604 may be positioned within the lumen 326. In some implementations, the balloon catheter 604 may include a balloon 610 positioned at or a distance away from the distal end of the balloon catheter 604. In some implementations, the balloon 610 may have two configurations: one in which the cross-sectional diameter of the balloon 610 is less than the cross-sectional diameter of the lumen 326 and another in which the balloon 610 expands to have a cross-sectional diameter of the balloon 610 is greater than the cross-sectional diameter of the lumen 326. In some implementations, when the balloon 610 reaches the stent 618, the balloon 610 may expand causing the stent 618 to expand, creating a cavity within the stent 618 within the marrow 322. However, it should be understood that in some implementations, the awl 608 may have a window 318 instead of a stent 618, and the balloon 610 may expand from the window 318. In some implementations, the balloon 610 may be deployed once the balloon 610 reaches the stent 618 by elastic energy forcing the balloon 610 to expand using, for example, an elastically compressed material, spring, fold, and/or any other mechanism for creating elastic potential energy. In some implementations, the balloon 610 may fold outwardly into the stent 618. In some implementations, the balloon 610 may be remotely actuatable by a user such as a surgeon. In some implementations, the balloon 610 may be remotely actuatable by a user such as a surgeon by using a valve to compress one or more fluids in the lumen 326 and/or balloon catheter 604, causing the pressure of the one or more fluids in the lumen 326 and/or balloon catheter 604 to expand the balloon 610. In some implementations, the balloon 610 may expand uniformly relative to the cross-sectional circumference of the awl 608. In some implementations, the balloon 610 may be sized and/or shaped such that the balloon 610 may expand more into certain directions relative to the cross-sectional circumference of the awl 608. For example, in some implementations, the balloon 610 may have a larger radius in some parts of the balloon's 610 circumferential arc relative to the cross-sectional circumference of the awl 608 such that the balloon 610 may apply more pressure to and/or expand into specific portions of the marrow 322. In some implementations, a surgeon may prefer the balloon 610 to expand more in specific directions to direct the bone void filler 518 into the marrow 322 and away from sensitive areas outside of the bone 102. For example, in some implementations, the balloon 610 may expand with a larger diameter toward the portion of the bone 102 between the pedicles 202 and/or toward the side of the bone 102 adjacent to the pedicles 202. In some implementations, the balloon 610 may expand with a larger diameter toward the posterior direction.

In some implementations, when the awl 608 is being inserted into the marrow 322, a push rod (not shown) may be inserted into the awl 608 to prevent the stent 618 from collapsing while the awl 608 is inserted into the marrow 322. The push rod may also be used to block marrow from entering the stent 618. The push rod may later be removed to allow the stent 618 to expand. It should be understood that depending on the implementation, the push rod may be made of any material or materials including, for example, the same or different material or materials as the awl 608. In some implementations, the push rod may be made of a flexible material or materials.

Although FIG. 6 may illustrate an example of one spinal treatment system 152, in some implementations there may be a second spinal treatment system (not shown) which may mirror the spinal treatment system 152 about the sagittal plane. It should be understood that depending on the implementation, this second spinal treatment system may incorporate at least some or all of any of the aspects or features of any on the examples disclosed herein including, for example, the disclosure relating to at least FIGS. 3-8 and/or any disclosure relating to any unilateral or bilateral implementations.

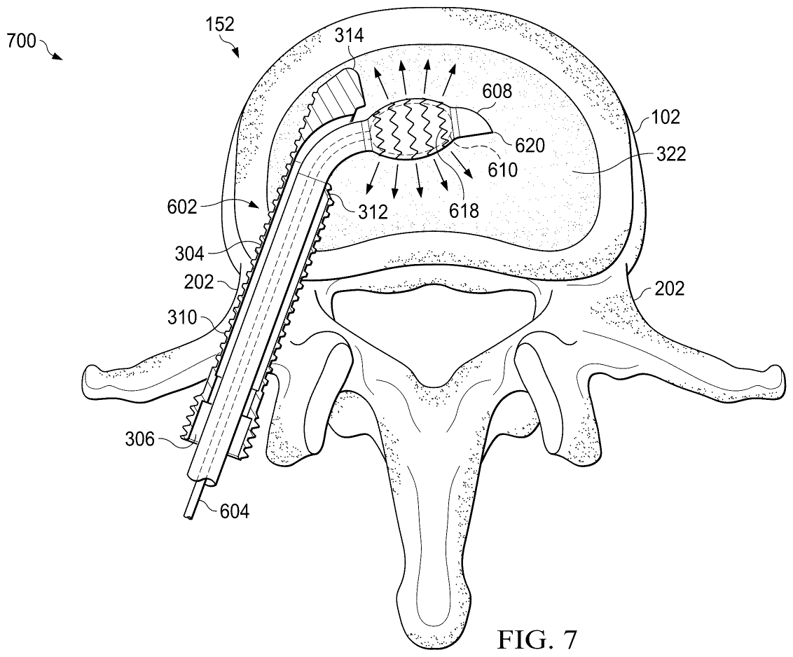

FIG. 7 illustrates a top cross-sectional view 700 of an example of a spinal treatment system 152 and a vertebra 102, according to some aspects of the present disclosure. Once the balloon 610 has been positioned adjacent to the stent 618, the balloon 610 may actuated causing the balloon 610 to expand the stent 618 into the marrow 322. In some implementations, the stent 618 may expand uniformly relative to the cross-sectional circumference of the awl 608. In some implementations, the stent 618 may be sized and/or shaped such that the stent 618 may expand more into certain directions relative to the cross-sectional circumference of the awl 608. For example, in some implementations, the stent 618 may have a larger expanded radius in some parts of the stent's 618 circumferential arc relative to the cross-sectional circumference of the awl 608 such that the stent 618 may apply more pressure to and/or expand specific portions of the marrow 322. In some implementations, a surgeon may prefer the stent 618 to expand more in specific directions to direct the bone void filler 518 into the marrow 322 and away from sensitive areas outside of the bone 102. For example, in some implementations, the stent 618 may expand with a larger diameter toward the portion of the bone 102 between the pedicles 202 and/or toward the side of the bone 102 adjacent to the pedicles 202. In some implementations, the stent 618 may expand with a larger diameter toward the posterior direction. It should be understood that depending on the implementation, the expanded shape of the stent 618 may be any size and/or shape including, for example, fully or substantially cylindrical, spherical, rectangular, square, triangular prismic, polygonal, a three-dimensional shape with a complexly shaped cross-section, and/or any other size and/or shape.

In some implementations, the balloon 610 and/or the balloon catheter may be removed from the awl 608 and/or transpedicular access port 304 once the stent has fully expanded. In some implementations, the balloon 610 and/or the balloon catheter 604 may remain inside the awl 608 and/or transpedicular access port 304 once the stent has fully expanded and/or at least a portion of the balloon and/or balloon catheter 604 may sealed in the awl 608 and/or transpedicular access port 304 with the cap 508.

In some implementations, the awl 308, 608, the stent 618, bone void filler 518, 818, or any other parts of the spinal treatment system 152 may be made of material or materials biologic and/or resorbable by the body over time including material or materials including, for example, polylactic acid (PLA), polyglycolic acid (PGA), polylactic-co-glycolic acid (PLGA), polydioxanone (PDO), polycaprolactone (PCL), magnesium, collagen, hyaluronic acid, alginic acid, gelatin, chitin, chitosan, carboxymethylcellulose (CMC), glycosaminoglycan, fibrinogen, tricalcium phosphate, carbonate apatite, any biodegradable material, and/or any other suitable material. In some implementations including at least partially biologic and/or resorbable material(s), the vertebral body bone may have the opportunity to remodel over time potentially leaving only native bone at the surgical site. Some implementations including at least partially biologic and/or resorbable material(s) may reduce load shielding, may improve radiographic visualization, and/or may increase options for surgical revision.

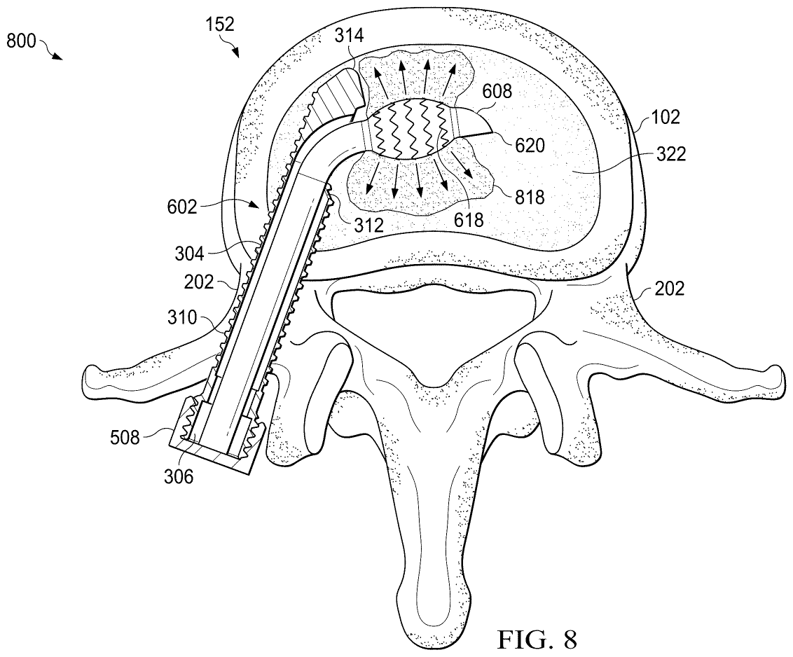

FIG. 8 illustrates a top cross-sectional view 800 of an example of a spinal treatment system 152 and a vertebra 102, according to some aspects of the present disclosure. In some implementations, once the balloon 610 and/or stent 618 are fully expanded, bone void filler 818 may be dispensed into the system. In some implementations, the surgeon may withdraw the balloon catheter 604 from the lumen 326. In some implementations, the surgeon may then insert bone void filler 818 into the lumen 326, and the bone void filler 818 may flow from the proximal end of the lumen 326, through the lumen 326, and out of the stent 618 into the marrow 322. In some implementations, the surgeon may insert a second catheter (not shown) into the lumen 326 which may dispense bone void filler 818 at or near the stent 618. In some implementations, the balloon 610 and/or stent 618 may clear some of the marrow 322 away from the stent openings before the bone void filler 818 is dispensed. This may allow the bone void filler 818 to flow into the marrow 322 more effectively.

In some implementations, once the bone void filler 818 has been dispensed into the bone marrow 322, the surgeon may seal the proximal end of the lumen 326 and/or cannula 306 with a cap 508. The cap 508 may prevent the bone void filler 818 from seeping from the proximal end of the lumen 326 and/or cannula 306 into the patient's body which could cause damage and/or discomfort to the patient's tissue. Additional implementations of the cap 508 may be discussed further herein.

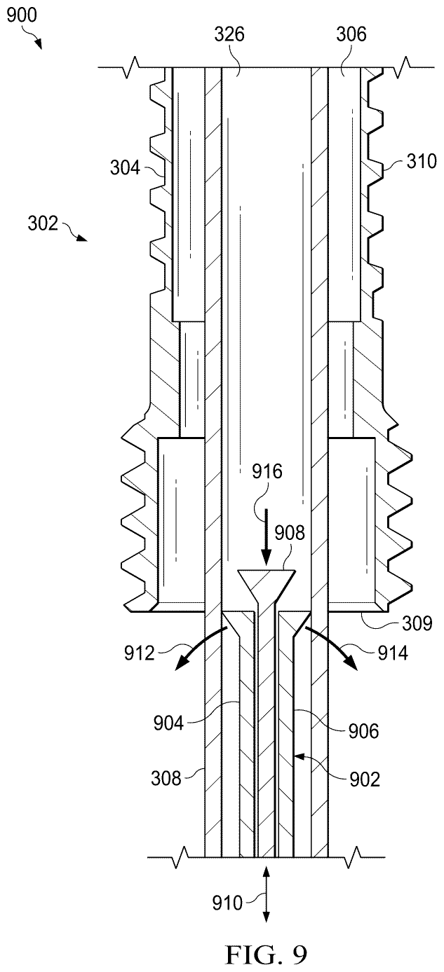

FIG. 9 illustrates a cross-sectional view 900 of an example of a cutter 902, according to some aspects of the present disclosure. In some implementations, the cutter 902 may include at least some or all of a first wedge 904, a second wedge 906, and a cam 908. The cutter 902 may severe the awl 308 at particular location.

In some implementations, the wedges 904, 906 may be positioned such that the lengthwise axes of the wedges 904, 906 are fully or substantially parallel. A wedge portion may extend outwardly at the distal ends of the wedges 904, 906. In some implementations, the wedge portions may have a fully or substantially wedged shape which may be sharp. It should be understood that the wedges 904, 906 may have any length including, for example, extending fully or substantially the entire length of the awl 308 or the fully or substantially the length of the awl extending from the cannula 306.

In some implementations, the cam 908 may be at least partially positioned between the wedges 904, 906 and may have a lengthwise axis fully or substantially parallel to the lengthwise axis of one or both of the wedges 904, 906. At least a portion of the cam 908 may be wider than the rest of the cam 908.

In some implementations, the cutter 902 may be used to trim the awl 308 and/or catheter 604 after the bone void filler 518, 818 is delivered. Trimming the awl 308 and/or catheter 604 may allow a cap 508 to seal the bone void filler 518, 818 into the cannula 306 and/or lumen 326 and/or prevent the bone void filler 518, 818 from seeping into other parts of the body and possibly causing tissue damage, pain, and/or discomfort. Before trimming, the cutter 902 may be inserted into the lumen 326. The awl 308 may be trimmed by the cutter 902 to be shorter than, the same length as, or longer than the transpedicular access port 304. In some implementations, the trimmed awl 308 may be fully or substantially flush with the proximal end 309. The cam 908 may be actuated by moving and/or translating the wedges 904, 906 in a proximal direction 910 which may cause the cam 908 to exert a proximally directed force 916 on the wedges 904, 906. The wedges 904, 906 and the cam 908 may both include opposing inclined planes which may lessen the force required to actuate wedges 904, 906. In response to the proximally directed force 916 of the cam 908, the cam 908 may exert outwardly directed forces 912, 914 on the wedges 904, 906, respectively. As the wedges 904, 906 move outwardly, the sharp portion of wedges 904, 906 may cut the adjacent portion of the awl 308 from inside the lumen 326.

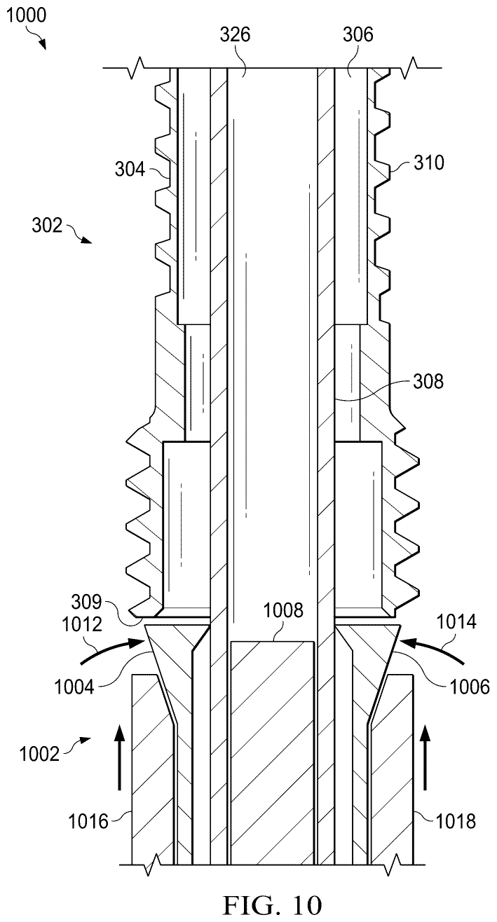

FIG. 10 illustrates a cross-sectional view 1000 of an example of a cutter 1002, according to some aspects of the present disclosure. In some implementations, the cutter 1002 may include at least some or all of a first wedge 1004, a second wedge 1006, a mandrel 1008, a first slider 1016, and a second slider 1018. The cutter 1002 may severe the awl 308 at particular location.

In some implementations, the wedges 1004, 1006 may be positioned such that the lengthwise axes of the wedges 1004, 1006 are fully or substantially parallel. A wedge portion may extend inwardly at the distal ends of the 1004, 1006. In some implementations, the wedge portions may have a fully or substantially wedged shape which may be sharp. It should be understood that the wedges 1004, 1006 may have any length including, for example, extending fully or substantially the entire length of the awl 308 or the fully or substantially the length of the awl extending from the cannula 306.

In some implementations, the mandrel 1008 may be at least partially positioned between the wedges 1004, 1006 and may have a lengthwise axis fully or substantially parallel to the lengthwise axis of one or both of the wedges 1004, 1006. In some implementations, the width of the mandrel may be fully or substantially consistent across at least the majority of the length of the mandrel 1008 and/or the mandrel 1008 may have a width and/or diameter less than or equal to the outer diameter of the lumen 326. It should be understood that depending on the embodiment, the cross-section of the mandrel 1008 may have any size and/or shape including, for example, circular, elliptical, triangular, rectangular, polygonal, a complex shape, and/or any other shape.

The sliders 1016, 1018 may be positioned at least partially adjacent to the outer surfaces of the wedges 1004, 1006. The sliders 1016, 1018 may be positioned such that the lengthwise axes of the sliders 1016, 1018 are fully or substantially parallel. In some implementations, the distal end of the sliders 1016, 1018 may be less wide than the rest of the sliders 1016, 1018. It should be understood that the sliders 1016, 1018 may have any length including, for example, extending fully or substantially the entire length of the awl 308 or the fully or substantially the length of the awl extending from the cannula 306.

In some implementations, the cutter 1002 may be used to trim the awl 308 and/or catheter 604 after the bone void filler 518, 818 is delivered. Trimming the awl 308 and/or catheter 604 may allow a cap 508 to seal the bone void filler 518, 818 into the cannula 306 and/or lumen 326 and/or prevent the bone void filler 518, 818 from seeping into other parts of the body and possibly causing tissue damage, pain, and/or discomfort. Before trimming, the mandrel 1008 may be inserted into the lumen 326 and/or at least a portion of the wedges 1004, 1006 may abut against the outer surface of the awl 308. The awl 308 may be trimmed by the cutter 1002 to be shorter than, the same length as, or longer than the transpedicular access port 304. The sliders 1016, 1018 may be actuated by moving and/or translating the sliders 1016, 1018 in distal direction which may cause the distal ends of the sliders 1016, 1018 to actuate the wedges 1004, 1006 and/or exert inwardly directed forces 1012, 1014 on the wedges 1004, 1006. The wedges 1004, 1006 and the sliders 1016, 1018 may both include opposing inclined planes which may lessen the force required to actuate wedges 1004, 1006. In response to the inwardly directed forces 1012, 1014 of the sliders 1016, 1018, the distal ends of the wedges 1004, 1006 may move inwardly towards the walls of the awl 308 and/or mandrel. As the wedges 1004, 1006 move inwardly, the sharp portion of wedges 1004, 1006 may cut the adjacent portion of the awl 308 from outside the lumen 326.

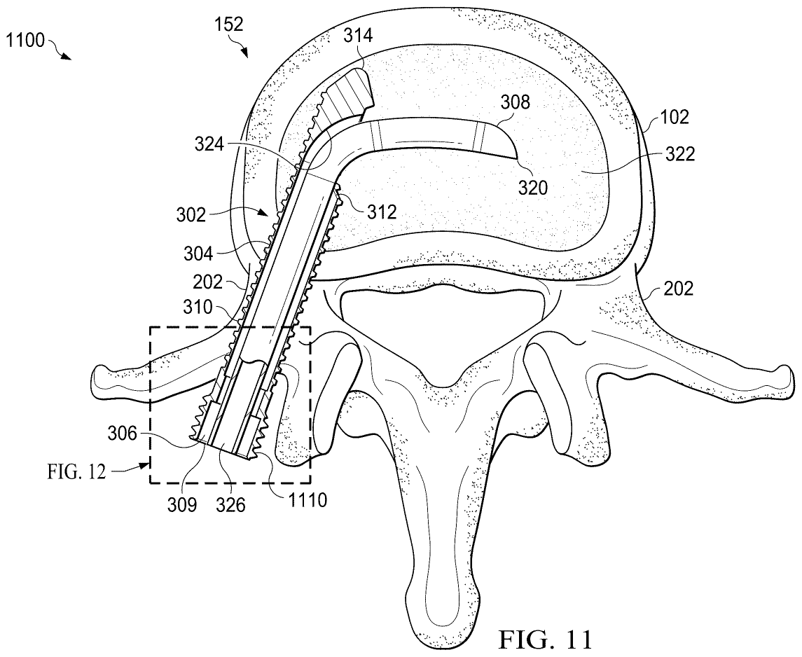

FIG. 11 illustrates a top cross-sectional view 1100 of an example of a spinal treatment system 152 and a vertebra 102, according to some aspects of the present disclosure. In some implementations, the transpedicular access port 304 may include second threads 1110. The second threads 1110 may be positioned at the proximal end 309 of the transpedicular access port 304. The second threads 1110 may extend a distance away from the proximal end 309. In some implementations, the second threads 1110 may have a different pitch than the threads 310 including, for example, a finer pitch than the threads 310. The second threads may be configured to interface with the cap 508.

In some implementations, the second threads 1110 may be used to interface instruments including, for example, a driver handle and/or drill bit. Thus, the surgeon may use the second threads to couple with and rotate the transpedicular access port 304.

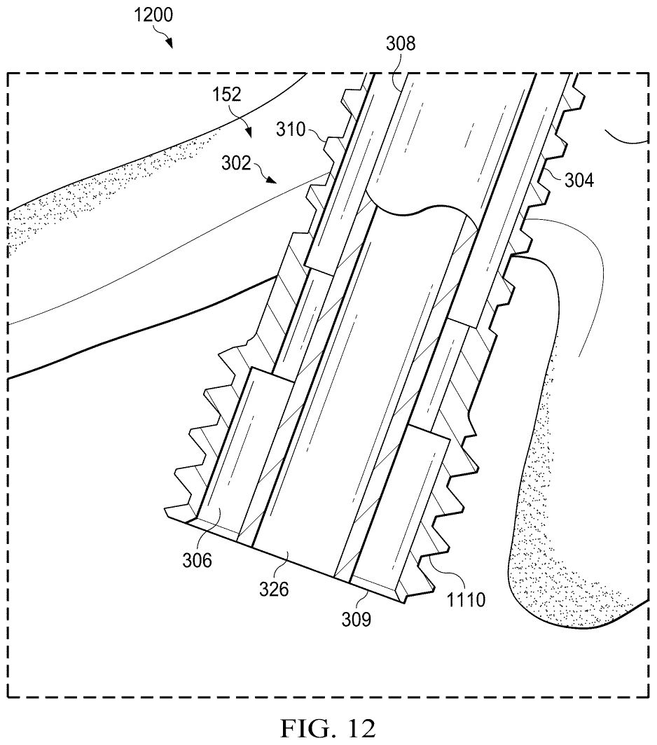

FIG. 12 illustrates a partial top cross-sectional view 1200 of an example of a spinal treatment system 152 and a vertebra 102, according to some aspects of the present disclosure. The example shown in FIG. 12 illustrates a partial and a more detailed view of implementations of the transpedicular access port 304 with second threads 1110.

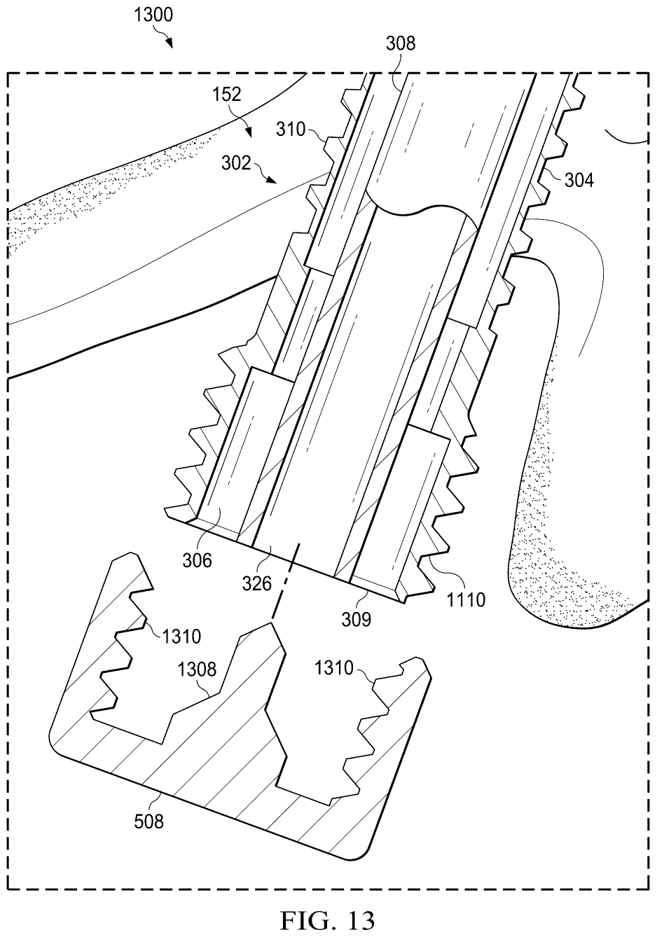

FIG. 13 illustrates a partial top cross-sectional view 1300 of an example of a spinal treatment system 152 and a vertebra 102, according to some aspects of the present disclosure. In some implementations, the transpedicular access port system 302 may include a cap 508. The cap 508 may include at least some or all of a projection or plug 1308 and cap threads 1310. The projection 1308 may extend from the distal facing end of the cap 508. In some implementations, at least a portion of the projection 1308 may have a cross-sectional diameter less than or equal to the diameter of the lumen 326 and/or cannula 306. In some implementations, at least a portion of the projection 1308 may have a cross-sectional diameter greater than or equal to the diameter of the lumen 326 and/or cannula 306. In some implementations, a portion of the projection 1308 may have a cross-sectional diameter less than or equal to the diameter of the lumen 326 and/or cannula 306 and another portion of the projection 1308 may have a cross-sectional diameter greater than or equal to the diameter of the lumen 326 and/or cannula 306. In some implementations, the projection 1308 may couple and/or interface with the lumen 326 with a compression fitting. However, it should be understood that depending on the implementation, the projection 1308 may couple and/or interface with the lumen 326 with any type of fit including, for example, an interference fit, a transition fit, a clearance fit, and/or any other type of fit.

The cap threads 1310 may be positioned on the inwardly facing surface(s) of the cap 508. The cap threads 1310 may cover a portion or the entire inwardly facing surface(s) of the cap 508. The cap threads 1310 may extend the entire or partial length (e.g., the length extending fully or substantially parallel to the axis extending from proximal to distal) of the cap 508. The cap threads 1310 may be the same or substantially the same length as the projection 1308 extends. In some implementations, the cap threads 1310 may interface with the second threads 1110 and/or the threads 310. Thus, in some implementations, the cap threads 1310 may have the same or substantially the same pitch as the second threads 1110 and/or the threads 310. The cap 508 may be positioned on the proximal end 309 by twisting the cap threads 1310 about the second threads 1110 and/or the threads 310. Thus, the cap 508 may seal the lumen 326, cannula 306, and/or catheter 604 which may prevent the bone void filler 518, 818 from seeping into the patient's body (e.g., extravasation). In some implementations, the cap 508 may secure the transpedicular access port 304 in place relative to awl 308 (or vice versa) and/or the cap 508 may lock the transpedicular access port 304 to the awl 308. In some implementations, the cap 508 may protect the second threads 1110 from wear so that the second threads 1110 could possibly be used again in future surgical procedures and/or revisions.

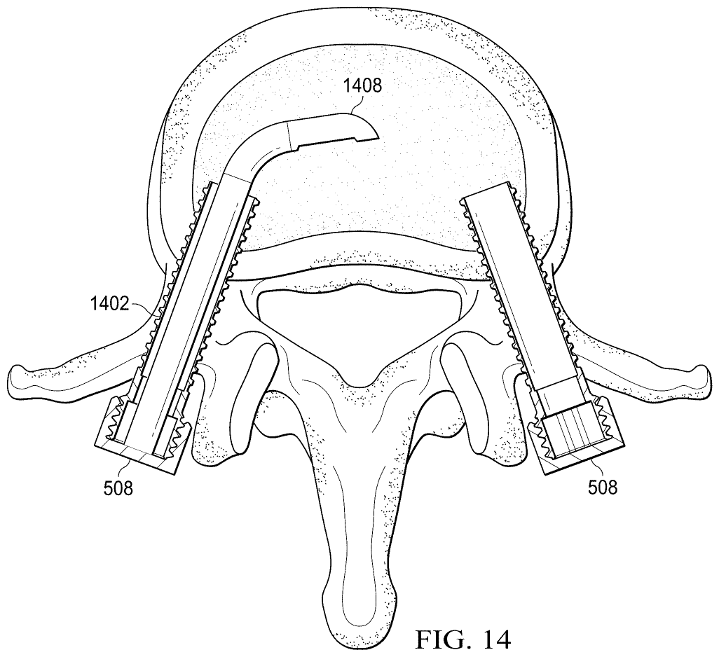

FIG. 14 illustrates still a further aspect of the present disclosure. In the illustrated embodiment, the delivery pin 1402 has a sufficient length to span the pedicle but does not extend fully into the cancellous bone of the vertebral body. An awl 1408, similar to that described above, is advanced along the axis of the delivery pin 1402 and exits a distal open end thereof. The awl includes a directional feature that controls the direction of the tip and/or curve of the awl body. In one aspect, the directional feature is one or more control wires within the awl that control tip direction. In another aspect, the directional feature is a generally rigid pre-bent push rod extending through the cannula of the awl and encouraging the awl to bend to follow the shape of the push rod. In some aspects, the one or more caps 508 may be not positioned on or may be removed from the delivery pin 1402 to allow access to the cannula of the delivery pin 1402, and then, in some aspects, the one or more caps 508 may screwed onto the threads of the delivery pin 1402 as shown at least in FIG. 14 to seal the cannula of the delivery pin 1402. In some aspects, the awl 1408 may remain in the delivery pin 1402 after the cap 508 is screwed onto the delivery pin 1402, while, in other aspects, the awl 1408 may be removed from the delivery pin 1402 before screwing the cap 508 onto the delivery pin 1402.

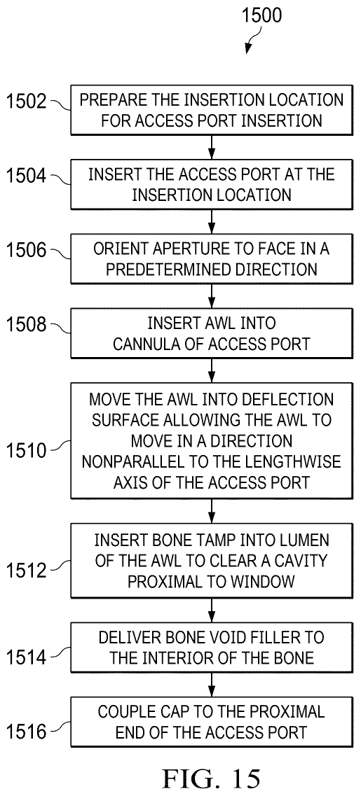

FIG. 15 illustrates an example of a method 1500 of stabilizing a vertebra 102, according to some aspects of the present disclosure. FIG. 15 may be an example method 1500 of delivering bone void filler 518, 818 to the interior of a vertebra 102 of a patient. At process 1502, the insertion location, such as the pedicle, may be prepared for transpedicular access port 304 insertion.

At process 1504, the transpedicular access port 304 may be inserted at the insertion location.

At process 1506, the aperture 312 may be oriented to face a predetermined direction, such as at least partially towards the sagittal plane.

At process 1508, the awl 308 may be inserted into the cannula 306 of the transpedicular access port 304.

At process 1510, the awl 308 may be moved into the deflection surface 324 allowing the awl 308 to move in a direction nonparallel to the lengthwise axis of the transpedicular access port 304.

At process 1512, the bone tamp 418 may be inserted to into the lumen 326 of the awl 308 to clear a cavity proximal to window 318. However, it should be understood that in some implementations, instead of or in addition to process 1512, a cavity may be cleared by a stent and/or balloon based on aspects and method discussed anywhere in this disclosure including, for example, disclosure relating to FIGS. 6-8 and/or FIG. 7.

At process 1514, bone void filler 518, 818 may be delivered to the interior of the bone 102. It should be understood that depending on the implementation, bone void filler 518, 818 may be delivered to the interior of the bone 102 based on aspects and method discussed anywhere in this disclosure including, for example, disclosure relating to FIGS. 3-8.

At optional process 1516, a cap 508 may be coupled to the proximal end 309 of the transpedicular access port 304.

It should be appreciated that any of the processes of method 1500 may be completed in any order. It should also be appreciated that some or all optional steps may be completed depending on the implementation. The order of the steps in method 1500 may be changed indiscriminately as bone void filler 518, 818 is delivered to the interior of the vertebra 102 of a patient.

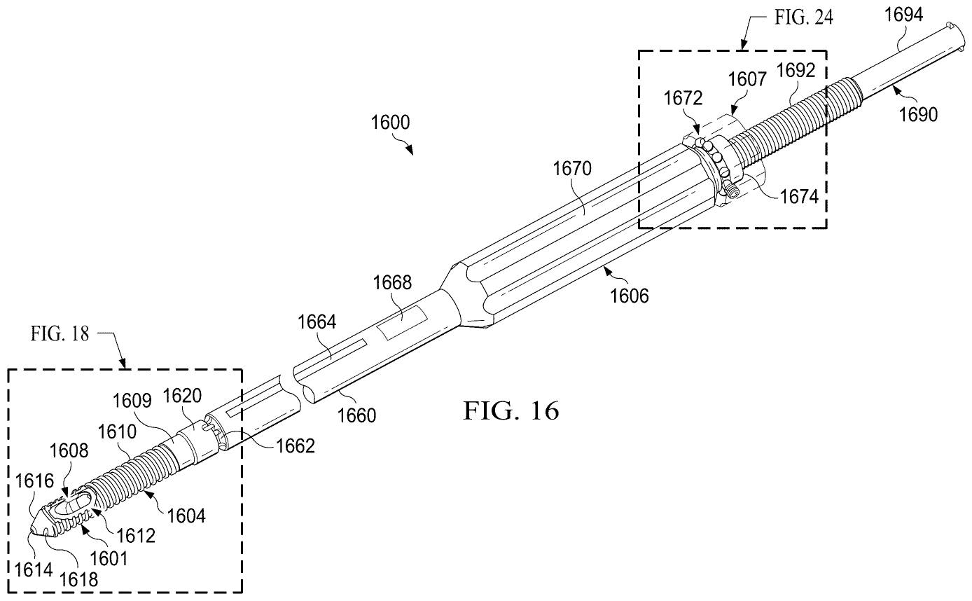

FIG. 16 illustrates a side perspective view of an example of a spinal treatment system 1600, according to some aspects of the present disclosure. It should be understood that the spinal treatment system 1600 may include any of the features of any of the spinal treatment systems discussed herein including, for example, the spinal treatment system 152 and/or the spinal treatment system 2500. The spinal treatment system 1600 may include a vertebral body access port system 1601. In some implementations, the vertebral body access port system 1601 may include any of a transpedicular access port 1604 and a flexible awl 1608. The transpedicular access port 1604 may also be referred to as an injection pin, an access port, a pin, a transpedicular screw, pedicle liner, pedicle working channel, an anchor, and/or any other suitable term. In addition to the transpedicular access port system 1601, the spinal treatment system 1600 may include any of a guide tube 1606, a knob 1607, and an inner shaft 1690.

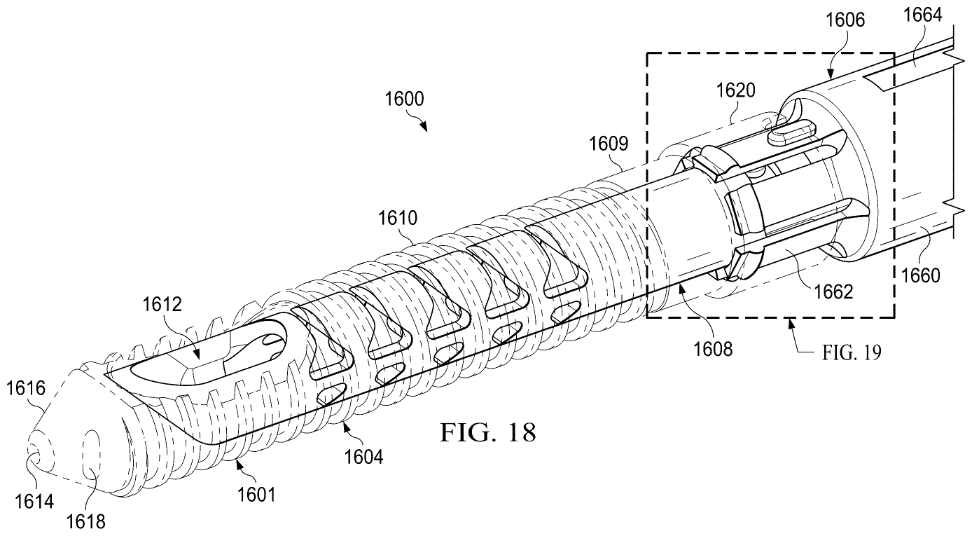

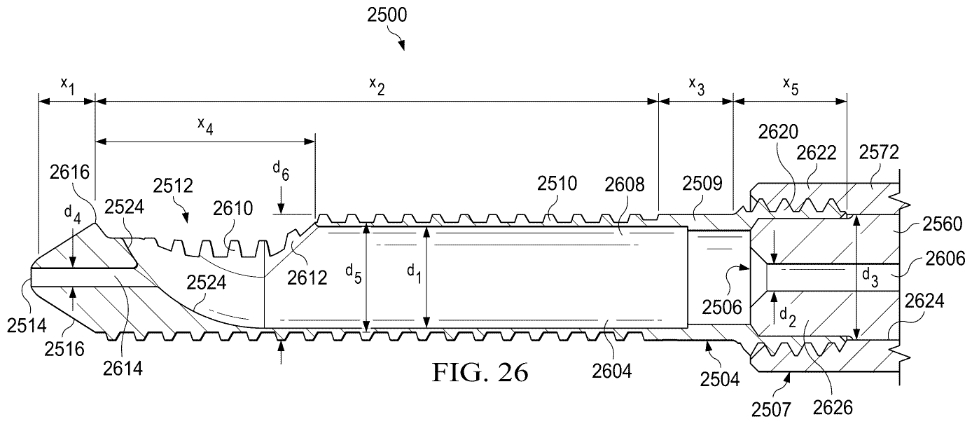

The transpedicular access port 1604 may include any of a tip 1614, a distal end 1616, a notch 1618, threads 1610, an aperture 1612 (working access aperture), a non-threaded portion 1609, and/or a proximal portion 1620. The transpedicular access port 1604 may be discussed further herein in reference to at least FIGS. 18 and 26.

The tip 1614 may be the furthest distal point of the transpedicular access port 1604. In some aspects, the tip 1614 may be entirely or substantially pointed to allow the tip 1614 to pierce through bone. In some aspects, the tip 1614 may be entirely, substantially, or partially rounded, domes, and/or having a fillet. The distal end 1616 may include the entire distal portion of the transpedicular access port 1604 and may include the tip 1614, the notch 1618, and aperture 1612. In some aspects, the distal end 1616 may be conically shaped and/or frustoconically shaped such that the diameter of the distal end 1616 decreases closer to the tip 1614. In some aspects, the distal end 1616 may be non-threaded such that the surface of the distal end 1616 is entirely, substantially, or partially smooth and/or does not include threads. In some aspects, the distal end 1616 may be threaded with the thread ending at or near the tip 1614. In some aspects, the distal end 1616 may be at least partially threaded with the thread ending at or near the distal edge of the threads 1610. Thus, in some aspects, the distal end 1616 may include at least a portion of the threads 1610.

In some aspects, the distal end 1616 and/or a combination of the distal end 1616 and the tip 1614 may include a cut out (not shown) which may allow the transpedicular access port 1604 to be self-tapping such that the transpedicular access port 1604 can make a hole in bone without a hole being drilled in the bone prior. In some aspects in which the transpedicular access port 1604 is self-tapping, any drilled and tapped bone without an egress pathway would be compressed outwardly. In some aspects in which the transpedicular access port 1604 is self-tapping, some drilled and tapped bone may enter the aperture 1612 and/or the working channel 2604 and/or may later be cleared out by the awl 1608. In some aspects, the notch 1618 may act as both an indicator of the orientation of the aperture 1612 and as a cut out for self-tapping.

In some aspects, the notch 1618 may inform the user(s) of the orientation of the aperture 1612 for a visual on fluoroscopy. However, in some aspects, other parts of the geometry of the distal end 1616 may inform the user(s) of the orientation of the aperture 1612 for a visual on fluoroscopy. It should be understood that, depending on the implementation, the notch 1618 may have any size and/or shape as long as the notch 1618 makes clear the orientation of the aperture 1612 on radiography and/or fluoroscopy. In some aspects, the notch 1618 may be located fully or substantially 90 degrees axially displaced from the aperture 1612. Thus, in some aspects in which the aperture 1612 is oriented generally parallel to the energy vector emitted from the imaging system, such as X-rays in a fluoroscopy system, the aperture 1612 may not be visible on the imaging system but the notch 1618 may then be visible. In this manner, the user may determine the direction and/or orientation of the aperture 1612 via the external imaging system without being able to view the aperture 1612 with the external imaging system. In some aspects, such as when the notch 1618 is positioned to be axially aligned with the aperture 1612, it will be appreciated that the direction of the aperture 1612 may be visible on external imaging by the lack of threads adjacent the aperture 1612. In this manner, the user may determine the direction of the aperture 1612 via the external imaging system.

In some aspects, the threads 1610 may extend a majority of the length of the transpedicular access port 1604. The threads 1610 may extend from the non-threaded portion of the distal end 1616 to the non-threaded portion 1609.

In some aspects, the transpedicular access port 1604 may include a non-thread portion 1609 positioned proximally to the threads 1610. However, it should be understood that, depending on the implementation, the threads 1610 may extend all the way to the proximal portion 1620 or to the proximal edge of the transpedicular access port 1604.

In some aspects, the proximal portion 1620 may have a diameter greater than the diameter of the non-threaded portion 1609 and/or the threads 1610 such that a step forms between the proximal portion 1620 and the non-threaded portion 1609 and/or the threads 1610. In some aspects, the proximal portion 1620 may be threaded while in other aspects, the proximal portion 1620 may be non-threaded. In some aspects, the proximal portion 1620 may include an inner surface (not shown) which may interface with the guide tube 1606.

The guide tube 1606 may include any of a guide tube body 1660, a flange 1662, an indicator line 1664, an indicator label 1668, and/or a handle 1670. The guide tube 1606 may be hollow, tubular, and/or cannulated such that a passage extends from the distal end to the proximal end of the guide tube 1606. The guide tube body 1660 may extend a majority of the longitudinal length of the guide tube 1606. The guide tube body 1660 may be entirely or substantially cylindrical and/or hollow and/or tubular.

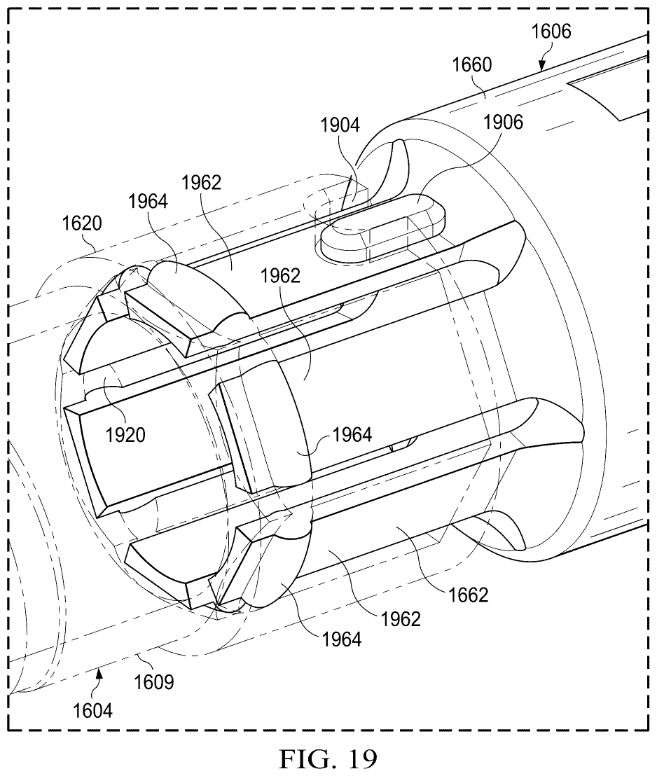

In some aspects, the flange 1662 may be inwardly bendable such that the proximal portion 1620 may extend over the flange 1662 and couple the transpedicular access port 1604 to the guide tube 1606. The proximal portion 1620, the flange 1662, and/or the interface between the transpedicular access port 1604 and the guide tube 1606 may be discussed further herein in regard to at least FIGS. 18 and 19.

The indicator line 1664 may be positioned to indicate the position of the aperture 1612. In some aspects, the flange 1662 may be structured to force the guide tube 1606 into alignment with the transpedicular access port 1604 such that the indicator line 1664 would be in alignment with the aperture 1612. In some aspects, the longitudinal axis of the indicator line 1664 may be entirely or substantially colinear or entirely or substantially parallel with the longitudinal axis of the aperture 1612.

In some aspects, an indicator label 1668 may be positioned adjacent and/or near to the indicator line 1664 to label the purpose of the indicator line 1664 as an indicator. In some aspects, the indicator label 1668 may say “SLOT”; however, it should be understood that, depending on the implementation, the indicator label 1668 may be any combination of characters and/or any way of labeling the indicator line 1664. The indicator line 1664 and/or the indicator label 1668 may be laser etched onto the guide tube 1606.

In some aspects, the guide tube 1606 may include a handle 1670 positioned at or near the proximal end 1674 of the guide tube 1606. In some aspects, the average diameter of the handle 1670 may be larger than the outer diameter of the guide tube body 1660 and/or the distal portion of the guide tube 1606. In some aspects, the handle 1670 may include a plurality of grooves to facilitate gripping by user(s). In some aspects, the handle 1670 may connect with the guide tube body 1660 at a sloped portion which extends between the differing diameters of the handle 1670 and the guide tube body 1660.

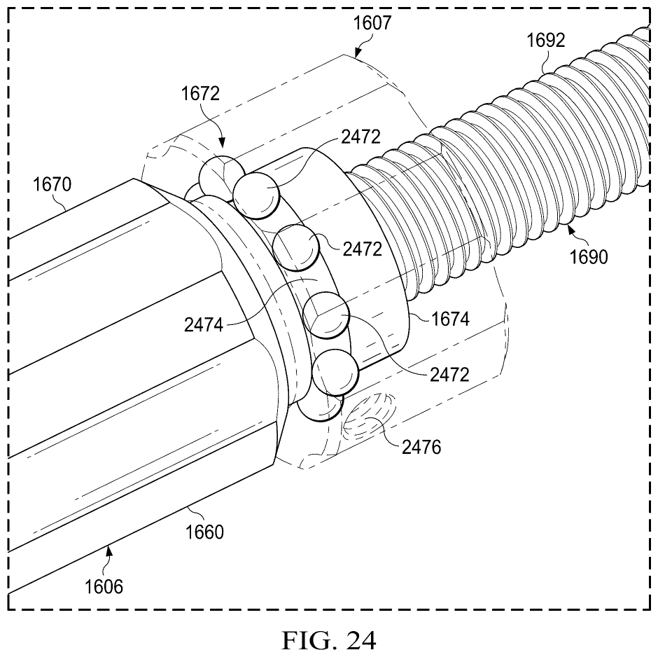

The knob 1607 may include a joint 1672 and may couple to the proximal end 1674 of the guide tube 1606. In some aspects, the joint 1672 may be a swivel, rotating, or pivoting joint. In some aspects, the joint 1672 may be a swivel, rotating, or pivoting joint with one or more ball bearings to reduce the friction of the joint 1672. The proximal opening of the knob 1607 may define the instrument engagement end of the spinal treatment system 1600 such that the working instruments of the surgeon may be inserted into the spinal treatment system 1600 though the instrument engagement end. The knob 1607 and the joint 1672 may be discussed further herein in reference to at least FIG. 24.

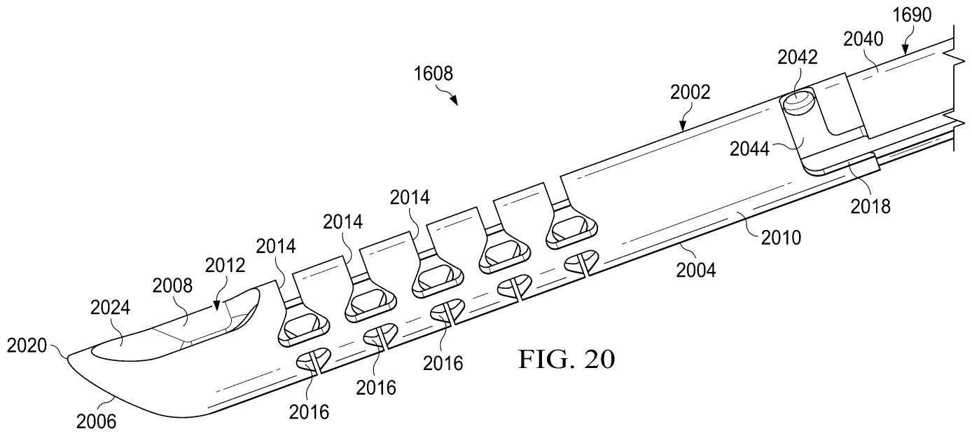

In some aspects, the awl 1608 may include an inner shaft 1690 which may include any of a threaded portion 1692 and a proximal portion 1694. In some aspects, the inner shaft 1690 may be hollow and/or tubular. The inner shaft 1690 may be at least partially threaded. The inner shaft 1690 may be discussed further herein in reference to at least FIGS. 20-22.

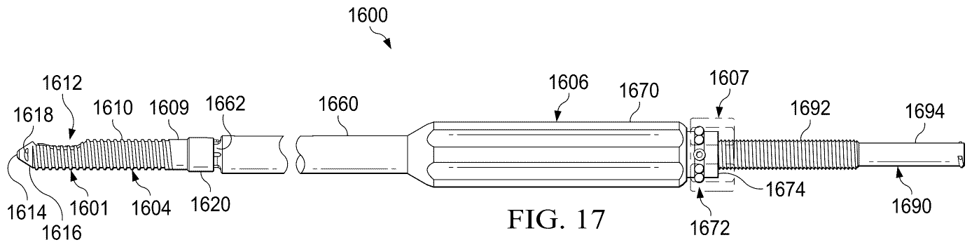

FIG. 17 illustrates a side view of an example of a spinal treatment system 1600, according to some aspects of the present disclosure.

FIG. 18 illustrates section view 18 of the example of a spinal treatment system 1600 in FIG. 16, according to some aspects of the present disclosure.

FIG. 19 illustrates section view 19 of the example of a spinal treatment system 1600 in FIG. 18, according to some aspects of the present disclosure. In some aspects, the flange 1662 may comprise one or more tabs 1962 extending from the guide tube body 1660. In some aspects, the one or more tabs 1962 may be spaced apart from each other and/or thinner than the wall of the guide tube body 1660 such that the one or more tabs 1962 may be bendable inwardly toward the central longitudinal axis of the guide tube 1606. In some aspects, some or all of the one or more tabs 1962 may include a ridge 1964 which may couple its corresponding of the one or more tabs 1962 with a groove 1920 on the inner surface of the proximal portion 1620. In some aspects, the one or more tabs 1962 may bias outwardly into the inner surface of the proximal portion 1620 securing the ridges 1964 within the groove 1920. In some aspects, the one or more tabs 1962 may be pushed outwardly to lock into the groove 1920. Thus, in some aspects, the one or more tabs 1962 may be forced outwardly with a locking rod (not shown) that will lock the drive interface together until the locking rod is removed such that the guide tube 1606 remains secured to the transpedicular access port 1604. In some aspects, the locking rod may be tubular to allow for the insertion of tools through the locking rod while the guide tube 1606 remains secured to the transpedicular access port 1604.

In some aspects, one of the tabs 1962 may include an additional tab 1906 which may align the guide tube 1606 with the transpedicular access port 1604 by being received within a notch 1904 formed on the access port 1604 as shown in FIG. 19. In some aspects, the longitudinal axis of the tabs 1906 and notch 1904 may be entirely or substantially colinear or entirely or substantially parallel with the longitudinal axis of the indicator line 1664 and/or when the guide tube 1606 is coupled to the transpedicular access port 1604, the tabs 1906 may be entirely or substantially colinear or entirely or substantially parallel with the longitudinal axis of the aperture 1612. In some aspects, the tabs 1906 engagement with the notch 1904 may restrict rotation of the guide tube 1606 about the guide tube's 1606 central longitudinal axis such that the indicator line 1664 is forced into alignment with the aperture 1612. In some aspects, the tabs 1906 may prevent the one or more ridges 1964 from rotating within the groove 1920. The tabs may couple with only a single slot or notch 1904 on the proximal portion 1620 of the transpedicular access port 1604 to ensure the axial alignment of the access port 1604 with the insertion tool, such as the guide tube 1606.

FIG. 20 illustrates a partial side perspective view of an example of an awl 1608, according to some aspects of the present disclosure. The awl 1608 may include any of a distal awl portion 2002 and the inner shaft 1690. In some aspects, the distal awl portion 2002 may be removably couplable to the inner shaft 1690. In some aspects, the distal awl portion 2002 may be made of more flexible, more elastic, and/or less rigid material than the inner shaft 1690.

The distal awl portion 2002 may include any of a distal awl portion body 2004, an outer surface 2010, an awl tip 2020, a lead surface 2006, a window 2012, a flute 2024, an inner lumen surface 2008, one or more compression cut outs 2014, one or more expansion cut outs 2016, and/or an L-shaped groove 2018.

The awl tip 2020 may be the most distal portion of the distal awl portion 2002. In some aspects, the awl tip 2020 may be sharp and/or sufficiently sharp to pierce bone and/or marrow. In some aspects, the awl tip 2020 may be made of a material harder or more rigid than the rest of the distal awl portion 2002. In some aspects, the awl tip 2020 may be rounded or may have a fillet.

In some aspects, the lead surface 2006 may be entirely or substantially flat. In some aspects, the lead surface 2006 may be at least partially curved convexly.

The inner shaft 1690 may include any of a peg 2042 and/or an interface surface 2044.

The peg 2042 may extend outwardly from the interface surface 2044. The peg 2042 may couple with the L-shaped groove 2018 to secure the inner shaft 1690 to the distal awl portion 2002. When the inner shaft 1690 and the distal awl portion 2002 are coupled together, the peg 2042 may be positioned in the portion of the L-shaped groove 2018 which extends laterally such that movement of the peg 2042 is restricted in either direction longitudinal to the awl 1608 (i.e., the direction the awl 1608 enters and the direction the awl 1608 exits). The peg 2042 allows the inner shaft 1690 to be removably coupled to the distal awl portion 2002. When the awl 1608 enters the guide tube 1606 and the transpedicular access port 1604, the peg 2042 can secure the inner shaft 1690 to the distal awl portion 2002. However, the peg 2042 can slide out from the L-shaped groove 2018 to leave the distal awl portion 2002 in place while the inner shaft 1690 is removed out the proximal end of the guide tube 1606. In some aspects, the distal awl portion 2002 may be removed in addition to the inner shaft 1690.

The interface surface 2044 may have an outer diameter less than the body 2040 of the inner shaft 1690 and/or an outer diameter less than the outer surface 2010 of the distal awl portion 2002 such that the distal awl portion 2002 and/or the L-shaped groove 2018 may overlap the interface surface 2044 when the inner shaft 1690 couples to the distal awl portion 2002. Thus, the top surface of the peg 2042 may be fully, substantially, or partially coplanar with the outer surface of the body 2040 of the inner shaft 1690.

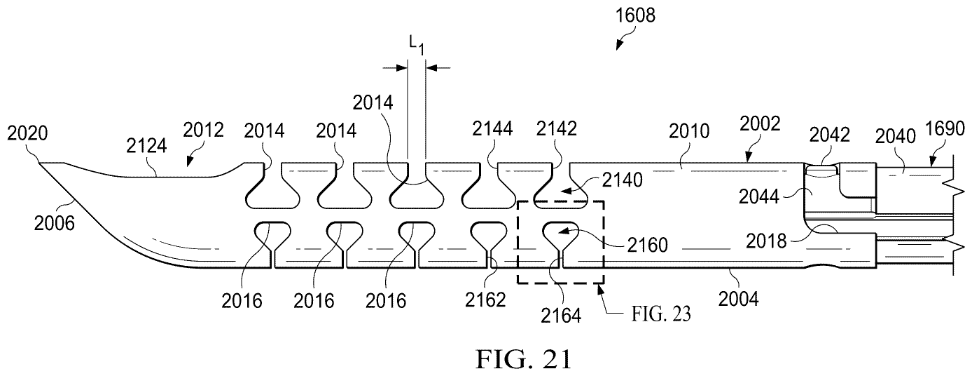

FIG. 21 illustrates a partial side view of an example of an awl 1608, according to some aspects of the present disclosure. Any of the one or more compression cut outs 2014 may have any of a dovetail 2140, a first abutment 2142, and a second abutment 2144.

While the compression cut outs 2014 in FIG. 21 are shown as dovetail shaped, it should be understood that, depending on the implementation, the compression cut outs 2014 may have any size and/or shape including, for example, circular, triangular, rectangular, polygonal, irregular, and/or any other suitable shape.

When the distal awl portion 2002 is straight as shown in FIG. 21, the first abutment 2142 may be spaced a distance L₁ from the second abutment 2144.

Any of the one or more expansion cut outs 2016 may have any of a dovetail 2160, a first surface 2162, and a second surface 2164.

While the expansion cut outs 2016 in FIG. 21 are shown as dovetail shaped, it should be understood that, depending on the implementation, the expansion cut outs 2016 may have any size and/or shape including, for example, circular, triangular, rectangular, polygonal, irregular, and/or any other suitable shape. In some aspects, the expansion cut outs 2016 may be smaller than the compression cut outs 2014 or vice versa.

In some aspects, when the distal awl portion 2002 is straight, for example, as shown in FIG. 21, the first surface 2162 may be spaced a distance apart from the second surface 2164 or may be spaced entirely or substantially adjacent and/or proximal to the second surface 2164.

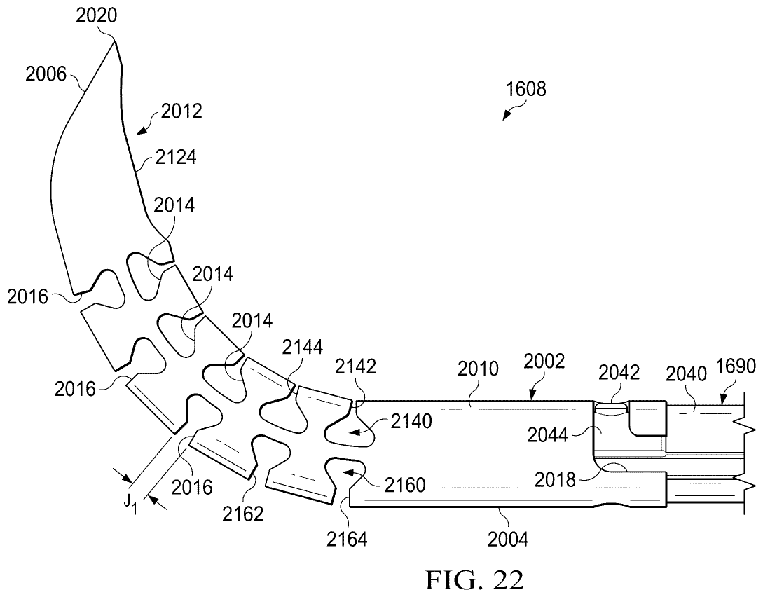

FIG. 22 illustrates a partial side view of an example of an awl 1608, according to some aspects of the present disclosure. As shown in FIG. 22, as the distal awl portion 2002 bends, the first abutment 2142 moves closer to the second abutment 2144 and the first surface 2162 move further away from the second surface 2164. In some aspects, L₁ (i.e., the distance between the first abutment 2142 and the second abutment 2144) may decrease the more that the distal awl portion 2002 bends. In some aspects, a distance J₁ (i.e., the distance between the first surface 2162 and the second surface 2164) may increase the more that the distal awl portion 2002 bends. In some aspects, the compression of the compression cut out(s) 2014 and/or the expansion of the expansion cut out(s) 2016 allows for the distal awl portion 2002 to bend while reducing the stress on the material of the distal awl portion 2002 as the distal awl portion 2002 bends. In some aspects, the compression of the compression cut out(s) 2014 and/or the expansion of the expansion cut out(s) 2016 allows for the distal awl portion 2002 to be made of a more rigid material than an awl without cut outs. Thus, the awl 1608 may be rigid enough to pierce bone and/or marrow but still sufficiently bendable to move through the aperture 1612.

In some aspects, the distal awl portion 2002 may bend to the point that L₁ becomes 0 or substantially 0 such that the first abutment 2142 contacts and/or bears against the second abutment 2144. In some aspects, the first abutment 2142 may not align with the second abutment 2144 such that L₁ decreases but does not become 0 and the first abutment 2142 does not contact the second abutment 2144.

It should be appreciated that, in some aspects, the compression of the compression cut out(s) 2014 and/or the expansion of the expansion cut out(s) 2016 may restrict or direct the flexion of the distal awl portion 2002 in only the desired direction. For example, in some aspects, the compression of the compression cut out(s) 2014 and/or the expansion of the expansion cut out(s) 2016 may restrict bending of the distal awl portion 2002 to only the direction shown in FIG. 22 or to only substantially in the direction shown in FIG. 22. In some aspects, the compression of the compression cut out(s) 2014 and/or the expansion of the expansion cut out(s) 2016 may restrict bending of the distal awl portion 2002 to only in the direction which the side, which includes the window 2012, faces. In some aspects, the compression of the compression cut out(s) 2014 and/or the expansion of the expansion cut out(s) 2016 may restrict bending of the distal awl portion 2002 to only in a direction which is entirely or substantially perpendicular to the direction the first abutment 2142 or the second abutment 2144 faces. It should be appreciated that in some aspects, only the expansion of the expansion cut out(s) 2016 causes the restriction of bending of the distal awl portion 2002 to one direction or substantially one direction because the compression cut out(s) 2014 are capable of expansion while, in some aspects, the expansion cut out(s) 2016 at least at a certain point are not capable of or not substantially capable of compression.

It should be appreciated that when bending of the awl 2708 is limited or substantially limited to one direction, the total force directed from the proximally exposed portion of the awl 2708 which may be transferred to the awl tip 2020 of the awl 2708 may be increased allowing the awl to more easily pierce bone and/or marrow.

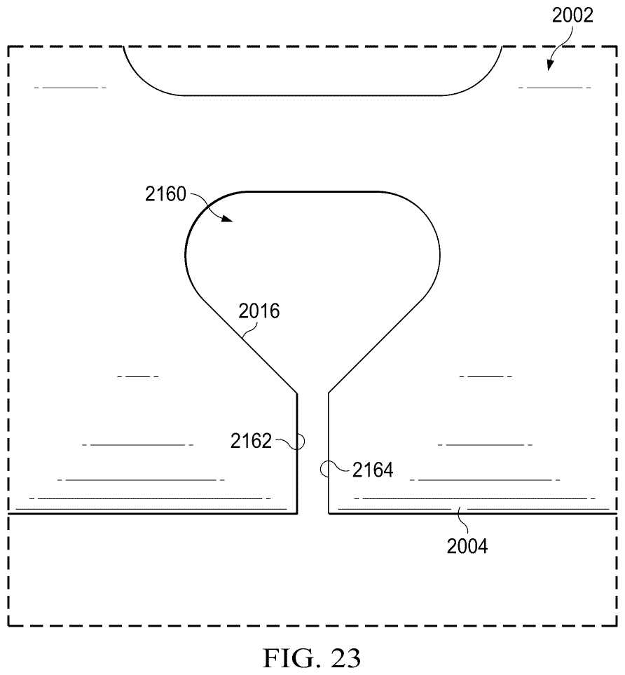

FIG. 23 illustrates section view 23 of the example of an awl 1608 in FIG. 21, according to some aspects of the present disclosure. In some aspects, when the distal awl portion 2002 is straight, for example, as shown in FIG. 21, the first surface 2162 may entirely, substantially, or partially contact and/or bear against the second surface 2164 as shown, for example, in FIG. 23. Thus, in some aspects, the separation between the first surface 2162 and the second surface 2164 may be manufactured by cutting the material between the first surface 2162 and the second surface 2164. In some aspects, even when the distal awl portion 2002 is straight as shown in FIG. 21, J₁ (i.e., the distance between the first surface 2162 and the second surface 2164 as shown in FIG. 22) may be greater than zero such that the first surface 2162 is spaced apart from the second surface 2164. It should be appreciated that in some aspects, in which the first surface 2162 may entirely, substantially, or partially contact and/or bear against the second surface 2164 as shown, for example, in FIG. 23, this prevents or substantially prevents the expansion cut out(s) 2016 from compression thus limiting or substantially limiting the direction of bending of the distal awl portion 2002.

FIG. 24 illustrates section view 24 of the example of a spinal treatment system 1600 in FIG. 16, according to some aspects of the present disclosure. In some aspects, the spinal treatment system 1600 may have a swivel joint with one or more ball bearings 2472 which may be at least partially positioned in a groove 2474 in the proximal end 1674 of the guide tube 1606 through threaded opening 2476 which may be closed and/or sealed by a threaded plug (not shown) and/or a set screw (not shown). In some aspects, the one or more ball bearings 2472 may reduce the friction to move the swivel joint. In some aspects, movement of the awl 1608 along the working channel 2604 is induced by twisting the knob 1607 relative to the guide tube 1606. The twisting of the knob 1607 causes the threads on the inner surface of the knob 1607 to interact with the threads on the inner shaft 1690 of the awl 1608 to cause linear movement of the awl 1608 along the working channel 2604. Rotating the knob 1607 in one direction (i.e., clockwise or counterclockwise) may cause the awl 1608 to move distally in the working channel 2604 and rotating the knob 1607 in the other direction may cause the awl 1608 to move proximally in the working channel 2604. It should be appreciated that, in some aspects, the rotational drive between the knob 1607 and the guide tube 1606 may impart an axial push force on the inner shaft 1690, which may keep the inner shaft 1690 to remain entirely or substantially in the same position and/or properly oriented such that the inner shaft 1690 does not rotate at all or does not substantially rotate. Thus, in some aspects, the lack of rotation of the inner shaft 1690 may prevent the peg 2042 from accidentally moving out of the L-shaped groove 2018 and accidentally detaching the inner shaft 1690 from the distal awl portion 2002.

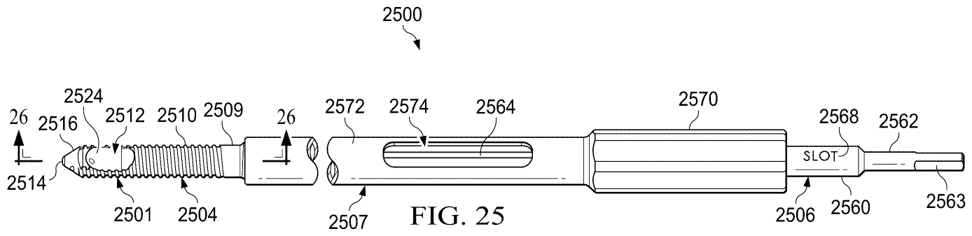

FIG. 25 illustrates a side view of an example of a spinal treatment system 1600, according to some aspects of the present disclosure. It should be understood that the spinal treatment system 2500 may include any of the features of any of the spinal treatment systems discussed herein including, for example, the spinal treatment system 152 and/or the spinal treatment system 1600. The spinal treatment system 2500 may include a transpedicular access port system 2501. In some implementations, the transpedicular access port system 2501 may include a transpedicular access port 2504. The transpedicular access port 2504 may also be referred to as an injection pin, an access port, a pedicle liner, a screw, a pedicle working channel, a pin, an anchor, and/or any other suitable term. In addition to the transpedicular access port system 2501, the spinal treatment system 2500 may include any of a guide tube 2506 and an outer sleeve 2507.

The transpedicular access port 2504 may include any of a tip 2514, a distal end 2516, a notch (not shown, refer to FIGS. 16-18), threads 2510, an aperture 2512, a non-threaded portion 2509, and/or a diverter 2524. The tip 2514 may be the furthest distal point of the transpedicular access port 2504. In some aspects, the tip 2514 may be entirely or substantially pointed to allow the tip 2514 to pierce through bone. In some aspects, the tip 2514 may be entirely, substantially, or partially rounded, domes, and/or having a fillet. The distal end 2516 may include the entire distal portion of the transpedicular access port 2504 and may include the tip 2514 and/or the notch. In some aspects, the distal end 2516 may be conically shaped and/or frustoconically shaped such that the diameter of the distal end 2516 decreases closer to the tip 2514. In some aspects, the distal end 2516 may be non-threaded such that the surface of the distal end 2516 is entirely, substantially, or partially smooth and/or does not include threads. In some aspects, the distal end 2516 may be threaded with the thread ending at or near the tip 2514.

In some aspects, the distal end 2516 and/or a combination of the distal end 2516 and the tip 2514 may include a cut out (not shown) which may allow the transpedicular access port 2504 to be self-tapping such that the transpedicular access port 2504 can make a hole in bone without a hole being drilled in the bone prior. In some aspects, the notch may act as both an indicator of the orientation of the aperture 2512 and as a cut out for self-tapping.

In some aspects, the threads 2510 may extend a majority of the length of the transpedicular access port 2504. The threads 2510 may extend from the non-threaded portion of the distal end 2516 to the non-threaded portion 2509.

In some aspects, the transpedicular access port 2504 may include a non-thread portion 2509 positioned proximally to the threads 2510. However, it should be understood that, depending on the implementation, the threads 2510 may extend all the way to the proximal edge of the transpedicular access port 2504. In some aspects, the non-threaded portion 2509 may include an inner surface (not shown) which may interface with the guide tube 2506.

The guide tube 2506 may include any of a guide tube body 2560, a proximal portion 2562, an indicator line 2564, and/or an indicator label 2568. The guide tube 2506 may be hollow, tubular, and/or cannulated such that a passage extends from the distal end to the proximal end of the guide tube 2506. The guide tube body 2560 may extend a majority of the longitudinal length of the guide tube 2506. The guide tube body 2560 may be entirely or substantially cylindrical and/or hollow and/or tubular. The interface between the transpedicular access port 2504 and the guide tube 2506 may be discussed further herein in regard to at least FIG. 26.

The indicator line 2564 may be positioned to indicate the position of the aperture 2512. In some aspects, the guide tube body 2560 may include an alignment feature such as a notch, bead, or tab, to force the guide tube 2506 into alignment with the transpedicular access port 2504 such that the indicator line 2564 would be in alignment with the aperture 2512. In some aspects, the longitudinal axis of the indicator line 2564 may be entirely or substantially colinear or entirely or substantially parallel with the longitudinal axis of the aperture 2512.

In some aspects, an indicator label 2568 may be positioned adjacent and/or near to the indicator line 2564 to label the purpose of the indicator line 2564 as an indicator. In some aspects, the indicator label 2568 may say “SLOT”; however, it should be understood that, depending on the implementation, the indicator label 2568 may be any combination of characters and/or any way of labeling the indicator line 2564.

In some aspects, the proximal portion 2562 of the guide tube 2506 may include a flat portion 2563. In some aspects, the proximal portion 2562 of the guide tube 2506 may have an outer diameter smaller than the outer diameter of the guide tube body 2560. The flat portion 2563 may be structured to be engaged by a tool (not shown) to grip, pull, and/or rotate the guide tube 2506.

The outer sleeve 2507 may include any of a view hole 2574 and/or a handle 2570. The outer sleeve 2507 may be hollow, tubular, and/or cannulated such that a passage extends from the distal end to the proximal end of the outer sleeve 2507. The outer sleeve body 2572 (i.e., the portion excluding the handle 2570) may extend a majority of the longitudinal length of the outer sleeve 2507. The outer sleeve body 2572 may be entirely or substantially cylindrical and/or hollow and/or tubular. The interface between the transpedicular access port 2504, the guide tube 2506, and/or the outer sleeve 2507 may be discussed further herein in regard to at least FIG. 26.

In some aspects, the inner surface of the cannula of the outer sleeve 2507 may have an inner diameter larger than the outer diameter of the outer surface of the guide tube body 2560 such that the outer sleeve 2507 may slide over the guide tube 2506. In some aspects, when the outer sleeve 2507 slides over the guide tube 2506 only the proximal parts and the portion of the guide tube 2506 visible through the view hole 2574 may be visible.

The view hole 2574 may be positioned in the outer sleeve body 2572. The view hole 2574 may allow the user(s) to view the indicator line 2564 to align the guide tube 2506 and/or the aperture 2512. In some aspects, the indicator label 2568 and/or at least a portion of the indicator line 2564 may be positioned on a proximal part of the guide tube body 2560 such that they are visible even when the outer sleeve 2507 is positioned over the guide tube 2506.

In some aspects, the outer sleeve 2507 may include a handle 2570 positioned at or near the proximal end of the outer sleeve 2507. In some aspects, the average diameter of the handle 2570 may be larger than the outer diameter of the outer sleeve body 2572 and/or the distal portion of the outer sleeve 2507. In some aspects, the handle 2570 may include a plurality of grooves to facilitate gripping by user(s). In some aspects, the handle 2570 may connect with the outer sleeve body 2572 at a sloped portion which extends between the differing diameters of the handle 2570 and the outer sleeve body 2572.

FIG. 26 illustrates a partial side cross-sectional view about line 26 of the example of a spinal treatment system 1600 in FIG. 25, according to some aspects of the present disclosure. The transpedicular access port 2504 may include any of a working channel 2604, an inner surface 2608, a distal cannula 2614, a ledge 2616, a diverter 2524, a transition surface 2610, a proximal sloped surface 2612, and a threaded portion 2620.

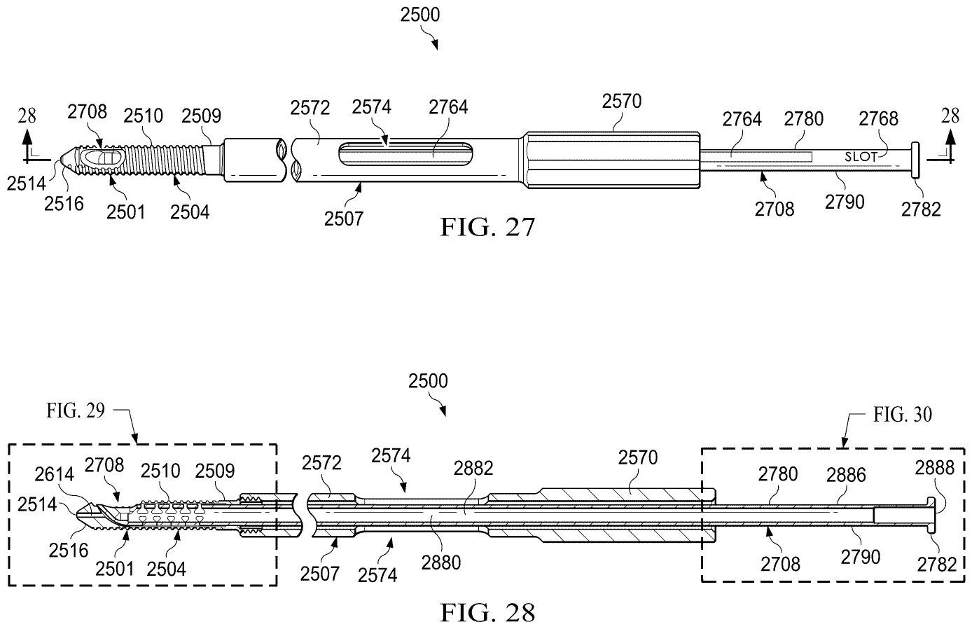

The working channel 2604 may extend at least the majority of the length of the transpedicular access port 2504. The working channel 2604 may be at least partially defined by the inner surface 2608. The diverter 2524 may be positioned at the proximal end of the working channel 2604. In some aspects, the diverter 2524 may divert an awl 2708, which is discussed further herein at least with respect to FIGS. 27-29, to bend from within the working channel 2604 to exit the working channel 2604 through the aperture 2512. In some aspects, the working channel 2604 does not extend the entire length of the transpedicular access port 2504. In some aspects, the working channel 2604 does not extend past the farthest distal portion of the aperture 2512. Thus, in some aspects, since the working channel 2604 does not extend the entire length of the transpedicular access port 2504, the awl 2708 is prevented from accidentally moving through the distal cannula 2614, which could be harmful for the patient, and is instead forced out of the aperture 2512. Thus, in some aspects, the distal end 2516 may be considered a closed end which may be free of instrument engagement. Instead, in some aspects, all instrument engagement will occur adjacent to the aperture 1612.

In some aspects, the slope between the furthest distal point of the diverter 2524 and the furthest proximal point of the diverter 2524 may be greater than 1 such that the diverter 2524 may be steeper and the awl 2708 may be steered more effectively in a perpendicular, substantially perpendicular, or partially perpendicular direction relative to the longitudinal axis of the transpedicular access port 2504 while reducing the amount of bending applied to the awl 2708.

In some aspects, the transpedicular access port 2504 may include a distal cannula 2614 which may have a diameter smaller than the diameter of the working channel 2604. In some aspects, the outer diameter of the awl 2708 may be smaller than the diameter of the working channel 2604 but larger than the diameter of the distal cannula 2614 such that the awl 2708 does not move through the distal cannula 2614 when the awl 2708 reaches the diverter 2524. The distal cannula may have a diameter large enough to fit a K wire or other guide wire (not shown) which may be used for initial positioning of the transpedicular access port 2504 within the bone. Thus, in some aspects, the diameter of the distal cannula may be large enough to fit a K wire but small enough to prevent the awl 2708 from going through the distal cannula 2614. It should be understood that, depending on the implementation and/or depending on the desired strength and flexibility need for a particular application, the size diameter of K wire and/or guide wire configured to fit in the distal cannula 2614 may be any size including, for example, 0.9 mm (0.035″), 1.1 mm (0.045″), 1.4 mm (0.054″), 1.6 mm (0.062″), and/or any other suitable diameter. Accordingly, in some aspects, the diameter of the distal cannula 2614, which may be referred to as d₄ herein, may be 0.01 to 0.5 mm larger than 0.9 mm (0.035″), 1.1 mm (0.045″), 1.4 mm (0.054″), 1.6 mm (0.062″), and/or any other diameter of a respective K wire and/or respective guide wire such that the K wire and/or guide wire may fit though the distal cannula 2614.

The aperture 2512 may extend from outside the transpedicular access port 2504 into the working channel 2604 such that the aperture 2512 opens the working channel 2604 to the outside of the transpedicular access port 2504. The aperture 2512 may be at least partially bounded on one side by the diverter 2524 and on another side by the proximal sloped surface 2612. In some aspects, the aperture 2512 may extend longitudinally across at least 2 threads of the transpedicular access port 2504, at least 3 threads of the transpedicular access port 2504, at least 4 threads of the transpedicular access port 2504, at least 5 threads of the transpedicular access port 2504, at least 6 threads of the transpedicular access port 2504, at least 7 threads of the transpedicular access port 2504, at least 8 threads of the transpedicular access port 2504, and/or any number of threads of the transpedicular access port 2504. Thus, in some aspects, the aperture 2512 may advantageously by large enough in longitudinal length to allow the awl 2708 to pass through without snagging or damaging the awl 2708. That is, in some aspects, the aperture 2512 is designed to allow the awl 2708 to pass through and is not small enough to restrain the awl 2708 from leaving the working channel 2604.

In some aspects, at least a portion of the transpedicular access port 2504 which defines the aperture 2512 that is at least partially cut out from the threads 2510 may include a transition surface 2610 and/or a proximal sloped surface 2612. The transition surface 2610 and/or the proximal sloped surface 2612 have a smooth intersection, an entirely or substantially flush transition, and/or an entirely or substantially continuous transition with each other and/or the inner surface 2608 so that the awl 2708 does not snag when passing through the aperture 2512. In some aspects, the transition surface 2610 and/or the proximal sloped surface 2612 may include a fillet and/or may be flared outwardly at the external edge to advantageously prevent or reduce the likelihood of the awl 2708 from snagging as the awl 2708 is deployed and/or retracted.

The guide tube 2506 may include a guide tube cannula 2606 and the outer sleeve 2507 may include any of an inner threaded portion 2622 and/or an outer sleeve cannula 2624. The threads of the inner threaded portion 2622 may couple with the threads of the threaded portion 2620 to couple the transpedicular access port 2504 to the outer sleeve 2507. In some aspects, the orientation (i.e., right-handed or left-handed) of the threads of the inner threaded portion 2622 may advantageously be the opposite of the direction of the threads on the transpedicular access port 2504 such that the transpedicular access port 2504 will not be loosened from the bone when outer sleeve 2507 is coupled and/or uncoupled from the transpedicular access port 2504.

In some aspects, when the transpedicular access port 2504 is secured to the outer sleeve 2507, the guide tube 2506 may be slid into the outer sleeve cannula 2624 from the proximal side of the outer sleeve 2507 until the guide tube abuts and/or is adjacent to the transpedicular access port 2504. In some aspects, a distal end 2626 of the guide tube 2506 may be shaped such that the guide tube 2506 is not rotatable or has limited rotation within the outer sleeve cannula 2624. In some aspects, the distal end 2626 of the guide tube 2506 may be shaped such that the guide tube 2506 is not rotatable or has limited rotation within the outer sleeve cannula 2624 and/or such that the indicator line 2564 must be aligned with the aperture 2512 for the guide tube 2506 to be inserted into the outer sleeve cannula 2624.

In some aspects, the outer sleeve cannula 2624 may have a diameter the same or substantially the same as that of the working channel 2604 such that the inner surface of the outer sleeve cannula 2624 may transition seamlessly to the inner surface 2608. In some aspects, the guide tube cannula 2606 may have a diameter smaller than the diameter of the working channel 2604. In some aspects, the guide tube cannula 2606 may have a diameter the same as or substantially the same as the diameter of the distal cannula 2614. In some aspects, the guide tube cannula 2606 may have a diameter larger than the diameter of the distal cannula 2614 but smaller than the diameter of the working channel 2604. In some aspects, the guide tube cannula 2606 may have a diameter of sufficient size to fit a K wire which may be used to position the transpedicular access port 2504 and/or the guide tube 2506 when the transpedicular access port 2504 is inserted into the bone. This, in some aspects, the central longitudinal axis of the guide tube cannula 2606 may be entirely or substantially aligned and/or colinear with the central longitudinal axis of the distal cannula 2614 when the guide tube 2506 is positioned within the outer sleeve cannula 2624.

In some aspects, for example as shown in FIG. 26, the longitudinal length of the non-threaded portion of the distal end 2516 may be x₁, the longitudinal length of the threads 2510 may be x₂, the longitudinal length of the non-threaded portion 2509 may be x₃, the longitudinal length of the aperture 2512 may be x₄, and/or the longitudinal length of the threaded portion 2620 may be x₅. In some aspects, x₁, x₃, x₄, and/or x₅ may each be less than x₂. In some aspects, the sum of x₁, x₂, x₃, and/or x₅ may be referred to as x (i.e., the total length of the transpedicular access port 2504). In some aspects, x₂ may constitute at least the majority of x. In some aspects, x₂ may constitute more than 75% of x. In some aspects, x₂ may constitute more than 80% of x. In some aspects, x₂ may constitute more than 90% of x. In some aspects, the longitudinal length of the distal end 2516 may be the sum of x₁ +x₄, and the central portion may be the sum of x₂ +x₃ less x₄. In some aspects, x₁ may be 5.5 mm or about 5.5 mm. In some aspects, x₂ may be 38 mm or about 38 mm. In some aspects, x₃ may be 4.5 mm or about 4.5 mm. In some aspects, x₄ may be 12 mm or about 12 mm. In some aspects, x₅ may be 7.25 mm or about 7.25 mm. It should be understood that the lengths discussed at least in this paragraph may be applied to any transpedicular access port discussed herein including, for example, at least transpedicular access port 304, transpedicular access port 1604, and/or transpedicular access port 2504.

In some aspects, for example as shown in FIG. 26, the diameter of the working channel 2604 may be d₁, the diameter of the guide tube cannula 2606 may be d₂, the diameter of the outer sleeve cannula 2624 may be d₃, the diameter of the distal cannula 2614 may be d₄, the outer root diameter of the transpedicular access port 2504 may be d₅, and/or the thread crest diameter of the transpedicular access port 2504 may be d₆. In some aspects, d₁ and/or d₃ may each be greater than each of d₂ and/or d₄. In some aspects, d₁ and/or d₃ may each be double the size of each of d₂ and/or d₄. In some aspects, d₁ and/or d₃ may each be 1.5 times the size of each of d₂ and/or d₄. In some aspects, d₁ and/or d₃ may each be 2.5 times the size of each of d₂ and/or d₄. In some aspects, d₁ may be equal to or substantially equal to d₃. In some aspects, d₂ may be equal to or substantially equal to d₄. In some aspects, d₁ may be less than d₃. In some aspects, d₂ may be greater than d₄. In some aspects, d₂ may be greater than d₄ but less than d₁ and/or d₃. In some aspects, d₁ may be more than half the size of d₅. In some aspects, d₁ may be 75% the size of d₅. In some aspects, d₁ may be 80% the size of d₅. In some aspects, d₁ may be 90% the size of d₅. In some aspects, d₁ may be 95% the size of d₅. In some aspects, d₁ may be 97% the size of d₅. It should be appreciated that the transpedicular access port 2504 may be thin walled such that d₁ is a large portion of d₅, since the transpedicular access port 2504 acts as a bone void filler delivery conduit and is not necessarily primarily intended to be load bearing to in a system inhibiting motion between adjacent vertebrae. By way of example, working channel diameter d₁ may be 8 mm while the root diameter d₅ is 10 mm, thereby creating an access port with a wall thickness of 1 mm. In a further embodiment, the thread crest diameter d₆ may be 12 mm, the working channel diameter d₁ may be 10 mm while the root diameter d₅ is 11 mm, thereby creating an access port with a wall thickness of 0.5 mm. In still a further, the thread crest diameter d₆ is 8 mm, the working channel diameter d₁ may be 6.5 mm while the root diameter d₅ is 7 mm, thereby creating an access port with a wall thickness of 0.25 mm. In some aspects, the transpedicular access port 2504 is primarily a delivery conduit and not primarily a load bearing structure like conventional pedicle screws. In some aspects, the thickness of the sidewall of the transpedicular access port 2504 (i.e., d₅-d₁) may only be able to resist between 50 lbs to 100 lbs of force (444.8 N) applied along the longitudinal axis of the transpedicular access port 2504. In some aspects, d₁ is sufficient size to permit the flow of a high viscosity (e.g., greater than 50 mPa·s) or super high viscosity (e.g., greater than 2,000 mPa·s) fluid or flowable material through the working channel 2604. In some aspects, d₁ may be sized large enough such that the working channel 2604 is unable to secure the transpedicular access port 2504 to a K wire and/or guide wire without the transpedicular access port 2504 moving and/or wobbling. In some aspects, d₁ may be 6 mm or about 6 mm. In some aspects, d₂ may be 3 mm or about 3 mm. In some aspects, d₃ may be 7.75 mm or about 7.75 mm. In some aspects, d₄ may be 2 mm or about 2 mm. In some aspects, d₅ may be 7.25 mm or about 7.25 mm. In some aspects, d₆ may be 8 mm or about 8 mm. It should be understood that the diameters discussed at least in this paragraph may be applied to any transpedicular access port discussed herein including, for example, at least transpedicular access port 304, transpedicular access port 1604, and/or transpedicular access port 2504.

FIG. 27 illustrates a side view of an example of a spinal treatment system 1600, according to some aspects of the present disclosure. FIG. 27 shows spinal treatment system 2500 with the guide tube 2506 removed and the awl 2708 added. Thus, the transpedicular access port system 2501 may include an awl 2708 when the guide tube 2506 is removed. The awl 2708 may include any of an awl body 2780, an indicator line 2764, an indicator label 2768, and/or a flange 2782. The awl body 2780 may extend from the distal end of the awl 2708 to the flange 2782.

The indicator line 2764 and/or the indicator label 2768 may include any of the features of indicator line 1664 and/or the indicator label 1668, respectively, and/or may include any of the features of indicator line 2564 and/or the indicator label 2568, respectively, as discussed herein.

The flange 2782 of the awl 2708 may be positioned at the proximal edge of the awl 2708. The diameter of the flange 2782 may be larger than the diameter of the awl body 2780 and/or the diameter of the outer sleeve cannula 2624. The flange 2782 may allow the user(s) to grab and remove the awl 2708.

FIG. 28 illustrates a side cross-sectional view about line 28 of the example of a spinal treatment system 1600 in FIG. 27, according to some aspects of the present disclosure. The awl 2708 may include any of a window 2812, a lumen 2880, a lumen surface 2882, a proximal end 2886, and/or a proximal opening 2888.

The awl 2708 may be hollow and/or tubular such that a lumen 2880 extends at least partially along the length of the awl 2708. The lumen 2880 may extend from the window 2812 to the proximal opening 2888. The lumen 2880 may be at least partially defined by a lumen surface 2882.

The awl may have a proximal end 2886 that comprises the portion of the awl 2708 which is outside the proximal opening of the outer sleeve cannula 2624 when the awl 2708 is at least partially inside the outer sleeve cannula 2624. At the proximal edge of the lumen 2880, the awl 2708 may have a proximal opening 2888 which provides access to the lumen 2880. The proximal opening 2888 may be used to supply flowable materials, such as bone void filler, to the lumen 2880 which may flow through the lumen 2880.

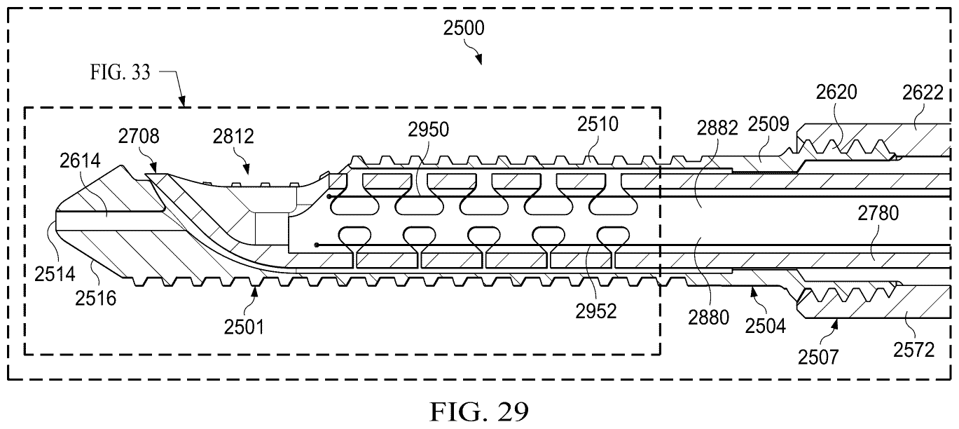

FIG. 29 illustrates section view 29 of the side cross-sectional view of the example of a spinal treatment system 1600 in FIG. 28, according to another aspect of the present disclosure. The flexible awl 2708 may include a pair of pull wires 2950 and 2952. Pull wire 2950 is joined to the distal end of the flexible awl adjacent the aperture, while pull wire 2952 is joined to the flexible awl adjacent the deflection tip. It will be appreciated that tension applied to pull wire 2950 while pull wire 2952 is slack will tend to bend the awl to close the spacing between the upper cut outs. Similarly, tension applied to the pull wire 2952 while pull wire 2950 is slack will tend to close gaps between the cutouts in the lower portion of the tubular member. A rotatable tensioning system can be applied to the insertion instrument adjacent knob 1607. Such tensioning mechanisms are known from guiding catheter assemblies.

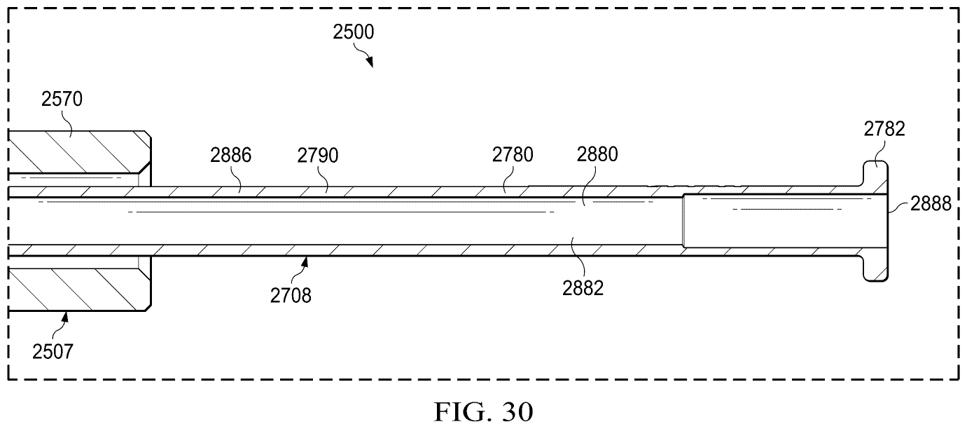

FIG. 30 illustrates section view 30 of the side cross-sectional view of the example of a spinal treatment system 1600 in FIG. 28, according to some aspects of the present disclosure.

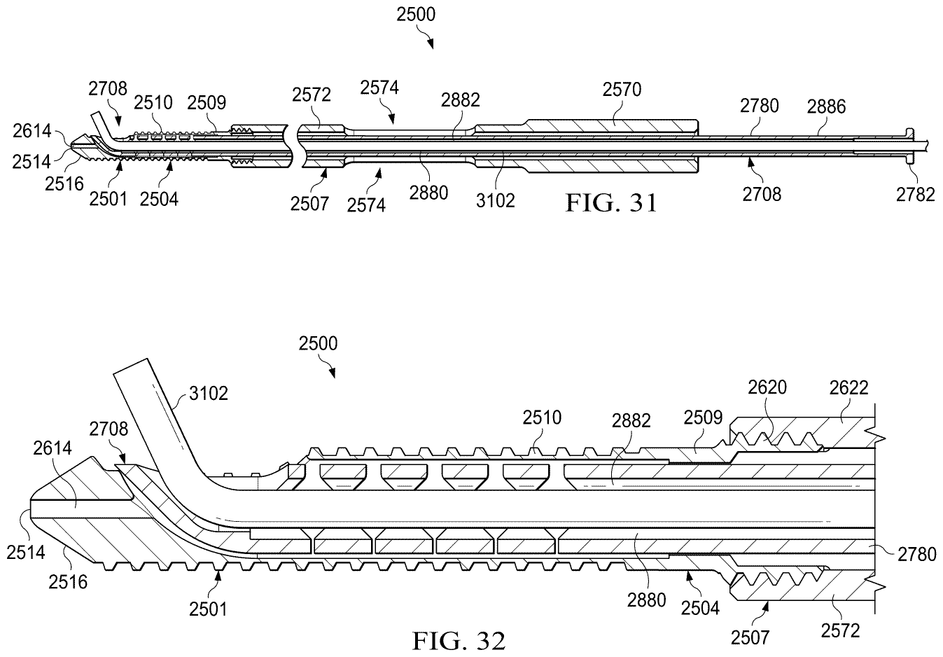

FIG. 31 illustrates the side cross-sectional view of the example of a spinal treatment system 1600 in FIG. 28 including an example of an injection tube, according to some aspects of the present disclosure. In some aspects, the spinal treatment system 2500 may include an injection tube 3102 for delivering fluid, such as bone void filler through the awl 2708. In some aspects, the outer diameter of the injection tube 3102 may be in the range of 2 mm to 4 mm. In some aspects, the inner diameter of the injection tube 3102 may be in the range of 1.5 mm to 3 mm. In some aspects, the inner diameter of the injection tube 3102 is sufficient size to permit the flow of a high viscosity (e.g., greater than 50 mPa·s) or super high viscosity (e.g., greater than 2,000 mPa·s) fluid or flowable material through the inner diameter of the injection tube 3102. In some aspects, d₁ is sufficient size to allow passage of an injection tube 3102 with an inner diameter of sufficient size to permit the flow of a high viscosity (e.g., greater than 50 mPa·s) or super high viscosity (e.g., greater than 2,000 mPa·s) fluid or flowable material through the inner diameter of the injection tube 3102. In some aspects, the inner diameter of the lumen 2880 is sufficient size to allow passage of an injection tube 3102 with an inner diameter of sufficient size to permit the flow of a high viscosity (e.g., greater than 50 mPa·s) or super high viscosity (e.g., greater than 2,000 mPa·s) fluid or flowable material through the inner diameter of the injection tube 3102. In some aspects, the inner diameter of the lumen 2880 is 3.6 mm or about 3.6 mm. In some aspects, the inner diameter of the lumen 2880 is 3.2 mm or about 3.2 mm. In some aspects, the inner diameter of the injection tube 3102 may be 2.69 mm (10 G, also known as 10 Gauge) or about 2.69 mm. In some aspects, the inner diameter of the injection tube 3102 may be 2.16 mm (12 G, also known as 12 Gauge) or about 2.16 mm. It should be appreciated that injection tubes with an inner diameter less than 2.16 mm (12 G) may be unable to allow for the flow of some high viscosity (e.g., greater than 50 mPa·s) or super high viscosity (e.g., greater than 2,000 mPa·s) fluids or flowable materials including, for example, Tetranite (bone adhesive), which may have a viscosity of more than 10,000 mPa·s. It should be appreciated that the working channel 2604 and the lumen 2880 have a sufficiently large diameter to allow for an injection tube 3102 with an inner diameter of more than 2.16 mm (12 G) which is sufficient to allow the flow of fluids or flowable materials including, for example, Tetranite (bone adhesive) through the injection tube 3102. Thus, it should be appreciated that an injection tube 3102 having an inner diameter greater than 2.16 mm would perform differently than an injection tube with a diameter less than 2.16 mm because the injection tube 3102 having an inner diameter greater than 2.16 mm would be able to transport higher viscosity bone adhesives, such as Tetranite.

In some aspects, the inner diameter of the working channel 2604, d₁, and/or the inner diameter of the lumen 2880 may be sized to receive an injection tubes 3102 of various different dimensions. For example, in some aspects, the inner diameter of the working channel 2604, d₁, and/or the inner diameter of the lumen 2880 may be sized to receive an injection tubes 3102 of various different dimensions based on the standard ISO 7864/ISO 10555 specifications for intravenous cannulas. In some aspects, d₅ may be exactly or about 7 mm, d₁ may be exactly or about 6 mm (therefore the wall thickness, d₅-d₁, may be exactly or about 0.5 mm), the outer diameter of the awl 2708 may be exactly or about 4.8 mm, the inner diameter of the awl 2708 may be exactly or about 3.2 mm (therefore the wall thickness of the awl 2708 may be exactly or about 0.8 mm), and thus, the spinal treatment system 1600, 2500 may be able to fit up to a 12 G injection tube 3102 with an outer diameter of exactly or about 2.77 mm and an inner diameter of exactly or about 2.16 mm. However, in some aspects, d₅, d₁, and/or the outer and inner diameter of the awl 2708 may be larger to accommodate a 10 G injection tube 3102 with an outer diameter of exactly or about 3.4 mm and an inner diameter of exactly or about 2.69 mm. In some aspects, d₅ may be exactly or about 7 mm, d₁ may be exactly or about 6 mm (therefore the wall thickness, d₅-d₁, may be exactly or about 0.5 mm), the outer diameter of the awl 2708 may be exactly or about 4.8 mm, the inner diameter of the awl 2708 may be exactly or about 3.2 mm (therefore the wall thickness of the awl 2708 may be exactly or about 0.8 mm), and thus, the spinal treatment system 1600, 2500 may be able to fit up to a 14G injection tube 3102 with an outer diameter of exactly or about 2.1 mm and an inner diameter of exactly or about 1.6 mm. It should be appreciated that a transpedicular access port 2504 with walls of very small thickness (e.g., about exactly or about 0.5 mm) will maximize the amount of space available for an injection tube 3102 in the average sized pedicle of the average sized patient. That is, in some aspects, the diameter of the average pedicle bone may be able to safely accommodate a transpedicular access port 2504 with an outer diameter, d₅, of at most 7 mm (8 mm screw crest diameter, d₆), which, in turn, limits the possible dimensions of the inner diameter of the working channel 2604, d₁. In some aspects, a transpedicular access port 2504 with a wall thickness of 0.5 mm may advantageously accommodate a 12 G injection tube 3102 with an exactly or about 2.16 mm inner diameter. However, it should be understood that in a larger patient with larger pedicle bones may accommodate a larger transpedicular access port 2504 with a larger outer diameter, d₅, but the same wall thickness (d₅ - d₁) of 0.5 mm such that the transpedicular access port 2504 may accommodate a 10 G injection tube with an exactly or about 2.69 mm inner diameter. It should be appreciated that, in some aspects, a 10 G injection tube 3102 may be ideal but a 12 G injection tube 3102 may be the largest gauge which could reasonably fit within the 7 mm outer diameter transpedicular access port 2504 that would fit within most pedicle anatomy. The size and ratios of the dimensions described in at least this paragraph are important to make sure the transpedicular access port 2504 is appropriately sized to not be too large for the pedicle while accommodating an appropriate sized delivery injection tube 3102 (All while making sure the appropriate wall thicknesses, d₅-d₁, exist for adequate strength for the transpedicular access port 2504 and awl 2708 flexibility). It should be appreciated that an injection tube 3102 having an inner diameter greater than 2.16 mm would perform differently than an injection tube with a diameter less than 2.16 mm because the injection tube 3102 having an inner diameter greater than 2.16 mm would be able to transport higher viscosity bone adhesives, such as Tetranite. Moreover, the present vertebral access system is not necessarily limited to transpedicular access, so larger size working access lumens may be utilized when the vertebral body is accessed via lateral, anterior or translateral approaches to the vertebral body.

FIG. 32 illustrates section view 32 of the side cross-sectional view of the example of a spinal treatment system 1600 in FIG. 31, according to some aspects of the present disclosure.

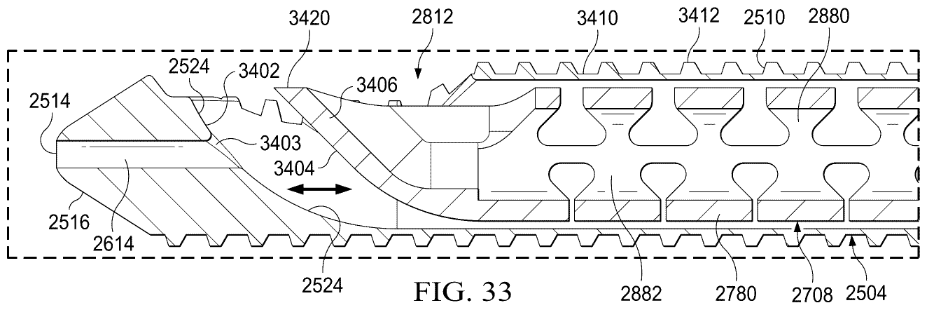

FIGS. 33-35 may illustrate the awl 2708 as it approaches and exits the aperture 2512. FIG. 33 illustrates a partial side cross-sectional view of an example of a spinal treatment system 1600, according to some aspects of the present disclosure.

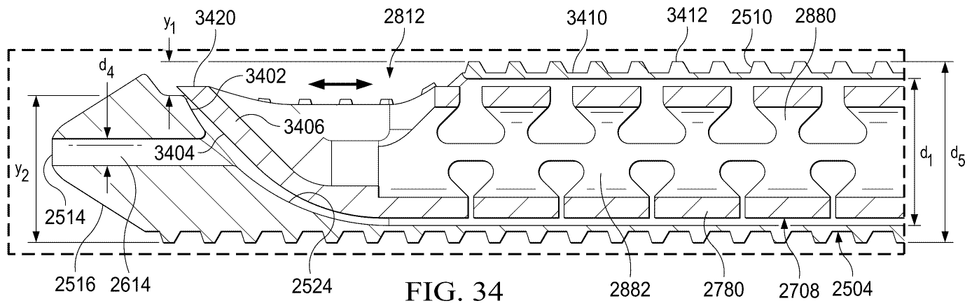

FIG. 34 illustrates section view 34 of the partial side cross-sectional view of the example of a spinal treatment system 1600 in FIG. 29, according to some aspects of the present disclosure. The transpedicular access port 2504 may include any of a set-back surface 3402, a fillet 3403, a thread root 3410, and/or a thread crest 3412.

In some aspects, the transpedicular access port 2504 may include a distal cannula 2614. In some aspects, the diverter 2524 may be structured to prevent the tip 3420 of the awl 2708 from snagging in the distal cannula 2614. Thus, in some aspects, the diverter 2524 may include a set-back surface 3402 which may be closer to the tip 2514 of the transpedicular access port 2504 then the portion of the diverter 2524 below the distal cannula 2614. Thus, in some aspects, if the tip 2820 of the awl 2708 drops forward into the distal cannula 2614 as the front end of the awl 2708 moves along the diverter 2524, the set-back surface may allow the awl 2708 to lurch forward into the distal cannula 2614 but still remain on course to exit through the aperture 2512. In some aspects, the front wall 3406 of the awl 2708 may be thicker at the top of the front wall 3406 than the bottom of the front wall 3406 such that a bearing surface 3404 of the awl 2708 contacts the set-back surface 3402 before any other part of the awl 2708 contacts the diverter 2524. In some aspects, the bearing surface 3404 may be flat or substantially flat. In some aspects, the bearing surface 3404 may be flat or substantially flat such that the bearing surface 3404 is not curved preventing or making the bearing surface 3404 less likely to snag on the distal cannula 2614 or any other part of the transpedicular access port 2504. In some aspects, the bearing surface 3404 being flat or substantially flat may increase the structural stability of the awl tip 3420 as it pierces bone and/or marrow by increasing the thickness of the front wall 3406 adjacent the awl tip 3420.

In some aspects, the fillet 3403 may be positioned at the opening between the working channel 2604 and the distal cannula 2614. The fillet 3403 may advantageously prevent or reduce the likelihood of the awl 2708 snagging on the distal cannula 2614 below the set-back surface 3402. For example, the fillet 3403 may advantageously prevent or reduce the likelihood of one or more of the one or more expansion cut outs 2016 of the awl 2708 from snagging on the distal cannula 2614 below the set-back surface 3402 when the awl 2708 is deployed and/or retracted.

In some aspects, the transpedicular access port 2504 may have an aperture distance, which may be referred to as y₁ and main body distance, which may be referred to as y₂. The sum of y₁ and y₂ may be d₆. The aperture distance, y₁, may be the distance of the cut out of the aperture 2512 when the transpedicular access port 2504 is viewed from cross section including, for example, the cross sections of FIGS. 33-35. In some aspects, y₁ may be less than half of d₅. In some aspects, y₁ may be more than 25% of d₅. In some aspects, y₁ may be less than 25% of d₅ but more than 10% of d₅. In some aspects, y₁ may be more than 10% of d₅. In some aspects, y₁ may be more than 5% of d₅. The aperture distance, y₁, may advantageously be a significant portion of the outer diameter of the transpedicular access port 2504, d₅, so that when the transpedicular access port 2504 is inserted, the aperture 2512 may collect bone and marrow within the working channel 2604 (that may be later cleared from the working channel 2604 by the awl 2708) which may open up a void in the bone in which bone void filler may be delivered.

It should be appreciated that in some implementations, the outer diameter, d₅, of the transpedicular access port 2504 may be in the range of 5 mm to 15 mm, which is larger than conventional pedicle screws. In some aspects, the transpedicular access port 2504 is thin walled and/or acts as bone void filler delivery conduit and is not necessarily primarily intended to be load bearing. In some aspects, the outer diameter, d₅, of the transpedicular access port 2504 may be in the range of 6 mm to 10 mm, which is larger than conventional pedicle screws. In some aspects, the outer diameter, d₅, of the transpedicular access port 2504 may be in the range of 7 mm to 9 mm, which is larger than conventional pedicle screws. In some aspects, the outer diameter, d₅, of the transpedicular access port 2504 may be about or exactly 8 mm, which is larger than conventional pedicle screws.

In some aspects, the outer diameter of the awl 2708 may be in the range of 3 mm to 9 mm. In some aspects, the diameter of the lumen 2880 may be about 2 mm to 6 mm.

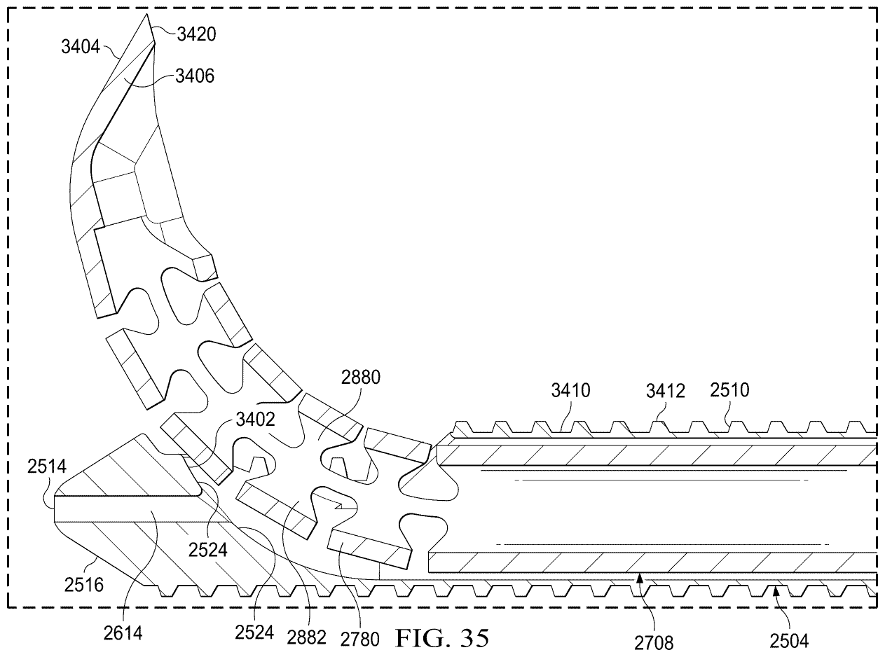

FIG. 35 illustrates a partial side cross-sectional view of an example of a spinal treatment system 1600, according to some aspects of the present disclosure.

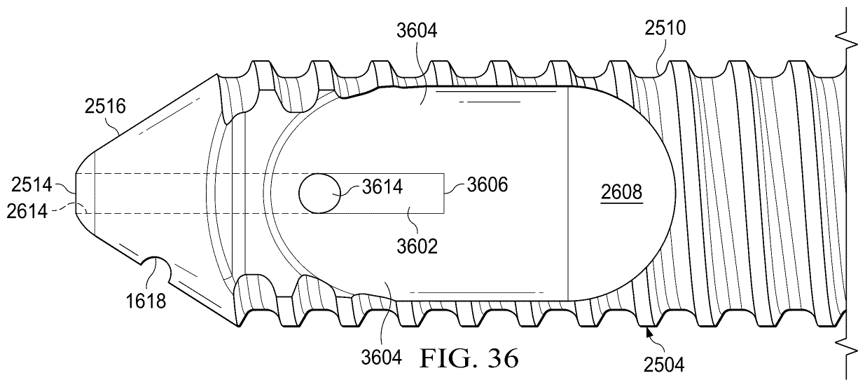

FIG. 36 illustrates a partial top view of an example of a spinal treatment system 1600, according to some aspects of the present disclosure. In some aspects, the transpedicular access port 2504 may include any of a trough 3602, diverter side surfaces 3604, a step 3606, and/or a cannula opening 3614. In some aspects, the trough 3602 may prevent the tip 2820 of the awl 2708 from snagging in the cannula opening 3614. Thus, in some aspects, the trough 3602 may work instead of or in conjunction with the set-back surface 3402 to prevent the tip 2820 of the awl 2708 from snagging in the cannula opening 3614. Since the awl 2708 is wider than the width of the trough 3602, when the awl 2708 contacts the diverter 2524, the diverter side surfaces 3604 of the diverter 2524 keep the awl 2708 elevated above the cannula opening 3614.

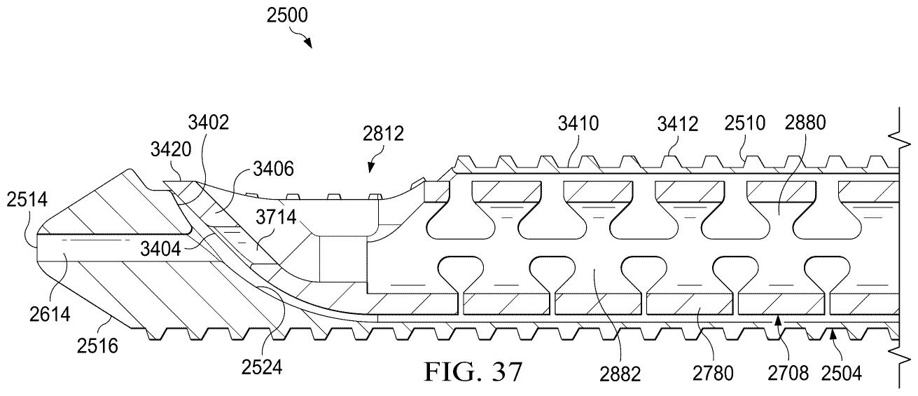

FIG. 37 illustrates a partial side cross-sectional view of an example of a spinal treatment system 1600, according to some aspects of the present disclosure. In some aspects, FIG. 37 shows an alternative implementation of the spinal treatment system 2500 in the same viewing perspective as FIG. 34. In some aspects, the awl 2708 may include a distal lumen 3714 which may allow the transpedicular access port 2504 to be inserted over a K wire such that the K wire is inserted through the distal cannula 2614, through the distal lumen 3714, and through the lumen 2880 to align the transpedicular access port 2504 with the target site. Thus, in some aspects, the spinal treatment system 2500 may be assembled with the awl 2708 preinstalled form a single use device incorporating the implantable working channel, awl, and insertion instrument into a single assembly. Once assembled with a guidewire extending within the distal cannula 2614 and lumen 3714, the guide wire will inhibit deployment of the awl through the side aperture thereby inhibiting accidental deployment of the awl outside of the working channel. In still a further aspect, the guidewire is pre-assembled within the distal cannula 2614 and the distal lumen 3714 before insertion into the patient. In this assembly, the guidewire may be extended beyond the tip 2514 of the distal end 2516 such that the guidewire may be advanced into the pedicle before the remaining assembly is inserted. Such an assembly reduces the number of steps that a surgeon must take relative to guidewire insertion followed by advancement of the implant assembly. In one aspect, the distal opening of lumen 3714 is at least 20% greater than the diameter of distal cannula 2614. In alternative configuration, the distal lumen 3714 has a diameter along its length that is at least 20% greater than the diameter of the cannula 2614. The larger opening and/or diameter of the lumen 3714 permits some misalignment between the cannula 2614 and lumen 3714 while still permitting the guidewire to pass through both opening making assembly, including intraoperatively, easier for a user.

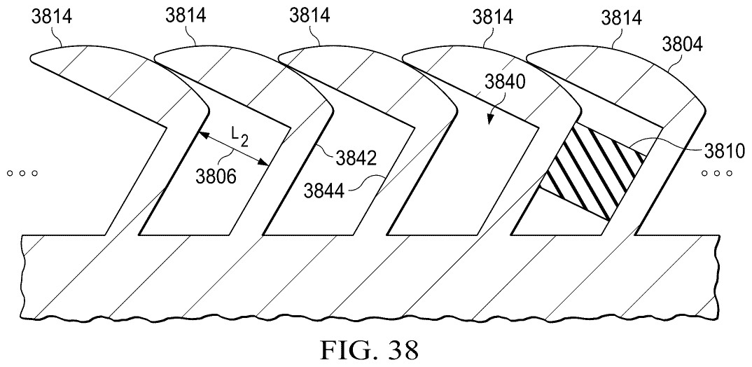

FIG. 38 illustrates a partial side cross-sectional view of an example of an awl 1608, 2708, according to some aspects of the present disclosure. In some aspects, instead of or in addition to the one or more compression cut outs 2014 as discussed at least with respect to FIGS. 20-23, the awl 2708 may have one or more scales 3814 which may overlap and/or allow for bending compression of the wall 3804 of the awl 2708. Some or all of the one or more scales 3814 may be separated from each other by a hole 3840. Some or all of the one or more scales 3814 may each have any of a first abutment 3842 and a second abutment 3844. The first abutment 3842 and the second abutment 3844 may at least partially define the hole 3840. In some aspects, when the distal awl portion 2002 is straight, for example, as shown in FIG. 21, the first abutment 3842 may be spaced from the second abutment 3844 by a compression distance 3806, which may be referred to as L₂. In some aspects, when the distal awl portion 2002 is at least partially bending, for example, as shown in FIG. 22, L₂ may decrease the more that the distal awl portion 2002 bends. In some aspects, the distal awl portion 2002 may bend to the point that the first abutment 3842 at least partially contacts and/or abuts the second abutment 3844. The one or more scales 3814 may advantageously allow the awl 2708 to bend while creating an at least partially continuous smooth point of contact for the fillet 3480, which is shown at least in FIGS. 33-35, to prevent snagging of the awl 2708 when the awl 2708 is withdrawn into the working channel 2604.

In some aspects, the awl 2708 may further include an elastic material 3810 which may be positioned to cover some or all of the holes 3840. The elastic material 3810 may be more elastic and/or less rigid than the material of the scales 3814 and/or the wall 3804 of the awl 2708. Thus, in some aspects, when the scales 3814 are compressed together, the elastic material 3810 will compress and allow the scales 3814 to move closer together. In some aspects, the elastic material 3810 may entirely, substantially, or partially cover some or all of the holes 3840. The elastic material 3810 advantageously allows the lumen 2880 to be fluid tight while still allowing the scales 3814 to compress.

It should be understood that, depending on the implementation, a similar structure as shown, for example, in FIG. 38 may be used instead of or in addition to the one or more expansion cut outs 2016 as discussed at least with respect to FIGS. 20-23. However, in at least this aspect, L₂ may be zero when the distal awl portion 2002 is straight, for example, as shown in FIG. 21, and L₂ may increase as the distal awl portion 2002 bends. In at least this aspect, the direction of the scales 3814 may advantageously be reversed to reduce snagging on, for example, the opening of the distal cannula 2614 and/or the ledge 2616 as the awl 2708 exits the transpedicular access port 2504.

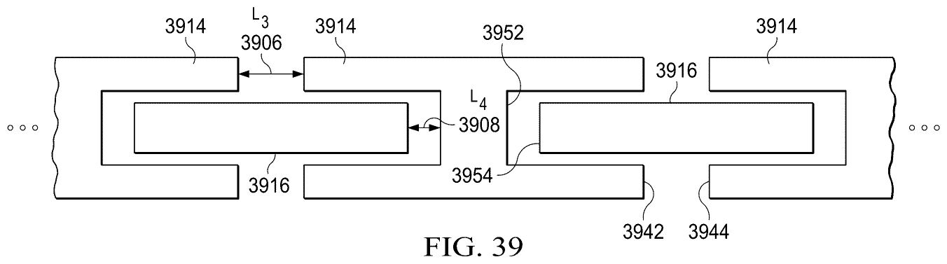

FIG. 39 illustrates a partial top view of an example of an awl 1608, 2708, according to some aspects of the present disclosure. In some aspects, instead of or in addition to the one or more compression cut outs 2014 as discussed at least with respect to FIGS. 20-23, the awl 2708 may have any of one or more first links 3914 and/or one or more second links 3916, which may allow for bending compression of the wall 3804 of the awl 2708. The first links 3914 may be separated from each other by a first compression distance 3906, which may be referred to L₃. The first links 3914 may each be separated from the second links 3916 by a second compression distance 3908, which may be referred to L₄. Some or all of the one or more first links 3914 may each have a first abutment 3942 and second abutment 3944. Some or all of the one or more first links 3914 may each have a third abutment 3952 and/or some or all of the one or more second links 3916 may each have a fourth abutment 3954. In some aspects, when the distal awl portion 2002 is straight, for example, as shown in FIG. 21, the first abutment 3942 may be spaced from the second abutment 3944 by a first compression distance 3906, which may be referred to as L₃. In some aspects, when the distal awl portion 2002 is straight, for example, as shown in FIG. 21, the third abutment 3952 may be spaced from the fourth abutment 3954 by a second compression distance 3908, which may be referred to as L₄. In some aspects, L₃ may be equal to or substantially equal to L₄. In some aspects, when the distal awl portion 2002 is at least partially bending, for example, as shown in FIG. 22, L₃ and/or L₄ may decrease the more that the distal awl portion 2002 bends. In some aspects, the distal awl portion 2002 may bend to the point that the first abutment 3942 at least partially contacts and/or abuts the second abutment 3944. In some aspects, the distal awl portion 2002 may bend to the point that the third abutment 3952 at least partially contacts and/or abuts the fourth abutment 3954. The one or more first links 3914 and/or the one or more second links 3916 may advantageously allow the awl 2708 to bend while creating an at least partially continuous smooth point of contact for the fillet 3480, which is shown at least in FIGS. 33-35, to prevent snagging of the awl 2708 when the awl 2708 is withdrawn into the working channel 2604. In some aspects, the portion of the first link(s) 3914 and/or the second link(s) 3916 at the middle of the longitudinal axis (i.e., farthest from the abutments) may be the widest (i.e., extending farthest in a direction out of the page of FIG. 39) such that a surface contacting the awl 2708 may advantageously be elevated above the gaps created by L₃ and/or L₄. In some aspects, the first link(s) 3914 may be coupled to each other and/or the second link(s) 3916 by any type of coupling structure including, for example, one or more pegs, an adhesive, one or more elastic materials (such as an elastic material 3810 discussed at least with respect to FIG. 38), and/or any other suitable structure.

In some aspects, the awl 2708 may further include an elastic material which may be positioned to cover some or all of the gaps between the first link(s) 3914 and/or the second link(s) 3916. The elastic material may be more elastic and/or less rigid than the material of the first link(s) 3914 and/or the second link(s) 3916 and/or the wall of the awl 2708. Thus, in some aspects, when the first link(s) 3914 and the second link(s) 3916 are compressed together, the elastic material will compress and allow the first link(s) 3914 and/or the second link(s) 3916 to move closer together. In some aspects, the elastic material may entirely, substantially, or partially cover some or all of gaps between the first link(s) 3914 and/or the second link(s) 3916. The elastic material advantageously allows the lumen 2880 to be fluid tight while still allowing the first link(s) 3914 and/or the second link(s) 3916 to compress.

It should be understood that, depending on the implementation, a similar structure as shown, for example, in FIG. 39 may be used instead of or in addition to the one or more expansion cut outs 2016 as discussed at least with respect to FIGS. 20-23. However, in at least this aspect, L₃ and/or L₄ may each be zero when the distal awl portion 2002 is straight, for example, as shown in FIG. 21, and L₃ and/or L₄ may increase as the distal awl portion 2002 bends. In at least this aspect, since the first link(s) 3914 and/or the second link(s) 3916 are fully or substantially symmetrical each along a line perpendicular to their longitudinal axis, the first link(s) 3914 and/or the second link(s) 3916 may advantageously reduce snagging on, for example, the opening of the distal cannula 2614 and/or the ledge 2616 as the awl 2708 travels the reverse direction and exits the transpedicular access port 2504.

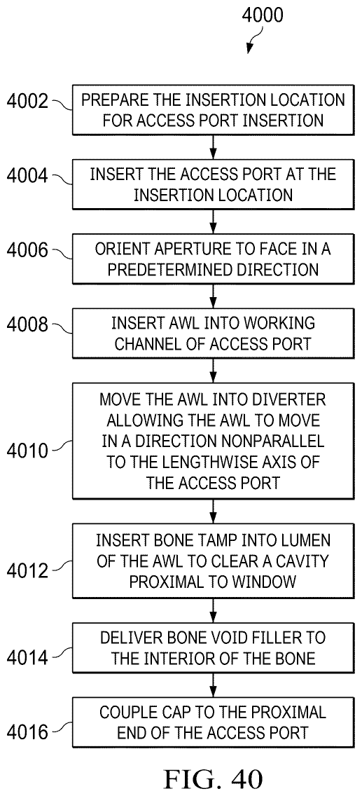

FIG. 40 illustrates an example of a method 4000 of stabilizing a vertebra, according to some aspects of the present disclosure. FIG. 40 may be an example method 4000 of delivering bone void filler 518, 818 to the interior of a vertebra 102 of a patient. It should be understood that the method 4000 may be applied to any aspects described herein including, for example, any aspects described in relation to at least FIGS. 16-24 and 31-39 as described herein. At process 4002, the insertion location, such as the pedicle, may be prepared for transpedicular access port 1604 insertion.

In some aspects, a K wire may be inserted at the insertion location which may be used to guide the transpedicular access port 1604 to the insertion location. Thus, in some aspects, the transpedicular access port 1604 may be cannulated to allow the transpedicular access port 1604 to be inserted over the K wire. However, it should be understood that in some aspects, the transpedicular access port 1604 may be aligned to the insertion location without using a K wire.

In some aspects, a trephine or Jamshidi needle may be used to prepare the insertion location prior to inserting a K wire or the transpedicular access port 1604.

At process 4004, the transpedicular access port 1604 may be inserted at the insertion location. In some aspects, a drill or other tool may be used to create a hole in the insertion location prior to inserting the transpedicular access port 1604. It should be understood that, depending on the implementation, any surgical drill may be used to create the hole in the insertion location for the transpedicular access port 1604 including, for example, a cannulated reamer and/or a cannulated drill. In some aspects, the diameter of the hole in the insertion location for the transpedicular access port 1604 may be about 5 mm to 10 mm in diameter.

In some aspects, transpedicular access port 1604 may be self-tapping such that the transpedicular access port 1604 may be inserted directly into the insertion location.

At process 4006, the access port 1604 is rotationally inserted with the outer threads causing longitudinal advancement into the vertebral body. The appropriate depth of the aperture 1612 is achieved which can be verified with depth markings and/or fluoroscopy. Once the appropriated depth within the vertebral body is achieved, the aperture 1612 may be oriented to face a predetermined direction, such as at least partially towards the sagittal plane. The direction of the aperture 1612 may be verified via fluoroscopy to identify the aperture and/or notch on the access port.

At process 4008, the awl 1608 may be inserted into the working channel 2604 of the transpedicular access port 1604. In some aspects, movement of the awl 1608 along the working channel 2604 is induced by twisting the knob 1607 relative to the guide tube 1606. The twisting of the knob 1607 causes the threads on the inner surface of the knob 1607 to interact with the threads on the inner shaft 1690 of the awl 1608 to cause linear movement of the awl 1608 along the working channel 2604. Rotating the knob 1607 in one direction (i.e., clockwise or counter-clockwise) may cause the awl 1608 to move distally in the working channel 2604 (deployment) and rotating the knob 1607 in the other direction may cause the awl 1608 to move proximally in the working channel 2604 (retraction). It should be appreciated that, in some aspects, the rotational drive between the knob 1607 and the guide tube 1606 may impart an axial push force on the inner shaft 1690, which may keep the inner shaft 1690 to remain entirely or substantially in the same rotational orientation and/or remain properly oriented such that the inner shaft 1690 does not rotate at all or does not substantially rotate. Thus, in some aspects, the lack of rotation of the inner shaft 1690 may prevent the peg 2042 from accidentally moving out of the L-shaped groove 2018 and accidentally detaching the inner shaft 1690 from the distal awl portion 2002.

At process 4010, the awl 1608 may be moved into the diverter 2524 allowing the awl 1608 to move in a direction nonparallel to the lengthwise axis of the transpedicular access port 1604. In some aspects, as the knob 1607 continues to be rotated and the awl 1608 moves forward in the working channel, the awl may eventually contact the diverter 2524 cause the awl to deflect and move out of the aperture 2512. In some aspects, the bearing surface 3404 of the awl 1608 may first contact the diverter 2524 at the set-back surface 3402 (i.e., above the distal cannula), which advantageously may prevent the tip 3420 of the awl 1608 from snagging in the distal cannula 2614. In some aspects, the bearing surface 3404 of the awl 1608 may first contact the diverter 2524 below the distal cannula 2614. In some aspects, the awl 1608 may be deployed, retracted, and re-deployed multiple times as the orientation and/or insertion depth of the transpedicular access port 1604 is adjusted to create bone voids followed by delivery of bone void filler to all desired locations. In embodiments including pull wires within the flexible awl, the pull wires may be tensioned to guide the tip of the awl to increase angulation for bone penetration in alternative directions or to reduce angulation to ease passage through the aperture as the flexible awl is withdrawn back into the access port.

It should be appreciated that many different parameters may be adjusted to change the location site of the bone void filler delivery. In some aspects, the angular position of the aperture 1612 relative to the longitudinal axis of the transpedicular access port 1604 may be precisely adjusted by rotating the transpedicular access port 2504 about its longitudinal axis and using the notch 1618 and aperture 1612 (on fluoroscopy), the indicator line 1664, and/or any other visual marker to determine the angular orientation of the aperture 1612. In some aspects, the insertion depth of the transpedicular access port 1604 may be adjusted to determine the position of the aperture 1612 relative to the longitudinal axis of the transpedicular access port 1604. In some aspects, the insertion depth and/or insertion angle of the awl 1608 into the bone may be adjusted using an adjustment structure including, for example, the knob 1607, which may deploy and retract the awl 1608. In some aspects, the radius of curvature of the bending of the awl 1608 may be adjusted by changing the dimension and/or shape of the compression cut out(s) 2014 and/or the expansion cut out(s) 2016. In some aspects, the spinal treatment system 1600 may include multiple different awls with compression cut out(s) 2014 and/or expansion cut out(s) 2016 of different dimensions and/or shapes to allow the surgeon to choose between different radius of curvatures of bending of the awl 1608. In some aspects, the bending of the awl 1608 may follow the path of a complex function instead of a partially circular radius of curvature. For example, in some aspects, the path of bending of the awl 1608 may be partially elliptical, parabolic, or hyperbolic. In some aspects, at least the parameters discussed in this paragraph and any other foreseeable parameters may be adjusted alone or in any combination to change to location site of bone void filler delivery. In some aspects, the surgeon(s) and/or user(s) may continuously or periodically monitor the transpedicular access port 1604 and/or the awl 1608 on fluoroscopy to determine the precise position of the transpedicular access port 1604 and/or the awl 1608.

At optional process 4012, the bone tamp 418 may be inserted to into the lumen 2880 of the awl 1608 to clear a cavity proximal to the window 2012. However, it should be understood that in some implementations, instead of or in addition to process 4012, a cavity may be cleared by a stent and/or balloon based on aspects and method discussed anywhere in this disclosure including, for example, disclosure relating to FIGS. 6-8 and/or FIG. 7.

At process 4014, bone void filler 518, 818 may be delivered to the interior of the bone 102. It should be understood that depending on the implementation, bone void filler 518, 818 may be delivered to the interior of the bone 102 based on aspects and method discussed anywhere in this disclosure including, for example, disclosure relating to FIGS. 3-8. In some aspects, as shown in FIGS. 31-32, for example, an injection tube 3102 may be inserted into the lumen 2880 of the awl 1608 in order to deliver bone void filler into the void space created by the awl 1608 and/or the bone tamp 418. In some aspects, the awl 1608 may be completely withdrawn from the working channel 2604 and/or the from the cannula of the guide tube 1606 and an injection tube 3102 may be inserted into the cannula of the guide tube 1606 and the working channel 2604 in order to deliver bone void filler into the void space created by the awl 1608 and/or the bone tamp 418. In some aspects, bone void filler may flow directly through the lumen 2880 of the awl 1608 in order to deliver bone void filler into the void space created by the awl 1608 and/or the bone tamp 418. In some aspects, the awl 1608 may be completely withdrawn from the working channel 2604 and/or the from the cannula of the guide tube 1606 and bone void filler may flow directly through the cannula of the guide tube 1606 and the working channel 2604 in order to deliver bone void filler into the void space created by the awl 1608 and/or the bone tamp 418. It should be appreciated that the awl 1608 may be either consumable or reusable such that, in some aspects, the awl 1608 may be implanted in the patient (i.e., may remain in the patient after surgery) while in other aspects, the awl 1608 may be removed and/or function only as an instrument. In some aspects in which the awl 1608 may remain in the patient after surgery, the inner shaft 1690 may be detached from the distal awl portion 2002 by moving the peg 2042 out of the L-shaped groove 2018 and withdrawing the inner shaft 1690 while the distal awl portion 2002 remains entirely or substantially in the same position.

At optional process 4016, a cap 508 may be coupled to the proximal portion 1620 of the transpedicular access port 1604 to seal the working channel 2604.

It should be appreciated that any of the processes of method 4000 may be completed in any order. It should also be appreciated that some or all optional steps may be completed depending on the implementation. The order of the steps in method 4000 may be changed indiscriminately as bone void filler 518, 818 is delivered to the interior of the vertebra 102 of a patient.

In some aspects, the method 4000 may be applied to any other aspects described herein including, for example, any aspects described in relation to at least FIGS. 25-39 as described herein. At process 4002, the insertion location, such as the pedicle, may be prepared for transpedicular access port 2504 insertion.

In some aspects, a K wire may be inserted at the insertion location which may be used to guide the transpedicular access port 2504 to the insertion location. Thus, in some aspects, the transpedicular access port 2504 may be cannulated to allow the transpedicular access port 2504 to be inserted over the K wire. However, it should be understood that in some aspects, the transpedicular access port 2504 may be aligned to the insertion location without using a K wire.

In some aspects, a trephine or Jamshidi needle may be used to prepare the insertion location prior to inserting a K wire or the transpedicular access port 2504.

At process 4004, the transpedicular access port 2504 may be inserted at the insertion location. In some aspects, a drill may be used to create a hole in the insertion location prior to inserting the transpedicular access port 2504. It should be understood that, depending on the implementation, any surgical drill may be used to create the hole in the insertion location for the transpedicular access port 2504 including, for example, a cannulated reamer and/or a cannulated drill. In some aspects, the diameter of the hole in the insertion location for the transpedicular access port 2504 may be about 5 mm to 10 mm in diameter.

In some aspects, transpedicular access port 2504 may be self-tapping such that the transpedicular access port 2504 may be inserted directly into the insertion location.

At process 4006, the aperture 2512 may be oriented to face a predetermined direction, such as at least partially towards the sagittal plane.

At process 4008, the awl 2708 may be inserted into the working channel 2604 of the transpedicular access port 2504. In some aspects, the awl 2708 may be driven forward along the working channel 2604 and into vertebral body through impaction of the flange 2782 at the proximal edge of the awl 2708. For example, a hammer or other percussion tool may be used to strike the flange 2782 at the proximal edge of the awl 2708 to drive the awl 2708. Thus, in some aspects, movement of the awl 2708 along the working channel 2604 is induced by impacting the awl 2708 to move relative to the outer sleeve 2507. This impaction may cause linear movement of the awl 2708 along the working channel 2604 which may include moving distally in the working channel 2604 (deployment) and moving proximally in the working channel 2604 (retraction). It should be appreciated that, in some aspects, the linear impaction of the awl 2708 may allow the awl 2708 to remain entirely or substantially in the same rotational orientation and/or remain properly oriented such that the awl 2708 does not rotate at all or does not substantially rotate. Thus, in some aspects, the lack of rotation of the awl 2708 may prevent the peg 2042 from accidentally moving out of the L-shaped groove 2018 and accidentally detaching the inner shaft 2790 from the distal awl portion 2002. In some aspects, the flange 2782 at the proximal edge of the awl 2708 may be used to grip the awl 2708 either with a tool or by hand to retract the awl 2708 from the work channel 2604 and/or the outer sleeve cannula 2624.

At process 4010, the awl 2708 may be moved into the diverter 2524 allowing the awl 2708 to move in a direction nonparallel to the lengthwise axis of the transpedicular access port 2504. In some aspects, as the awl 2708 is impacted and the awl 2708 is driven forward in the working channel, the awl may eventually contact the diverter 2524 cause the awl to deflect and move out of the aperture 2512. In some aspects, the bearing surface 3404 of the awl 2708 may first contact the diverter 2524 at the set-back surface 3402 (i.e., above the distal cannula), which advantageously may prevent the tip 3420 of the awl 2708 from snagging in the distal cannula 2614. In some aspects, the bearing surface 3404 of the awl 2708 may first contact the diverter 2524 below the distal cannula 2614. In some aspects, the awl 2708 may be deployed, retracted, and re-deployed multiple times as the orientation and/or insertion depth of the transpedicular access port 2504 is adjusted to create bone voids and deliver bone void filler to all desired locations.

It should be appreciated that many different parameters may be adjusted to change the location site of the bone void filler delivery. In some aspects, the angular position of the aperture 2512 relative to the longitudinal axis of the transpedicular access port 2504 may be precisely adjusted by rotating the transpedicular access port 2504 about its longitudinal axis and using the notch 1618 (on fluoroscopy), the indicator line 2564, and/or any other visual marker to determine the angular orientation of the aperture 2512. In some aspects, the insertion depth of the transpedicular access port 2504 may be adjusted to determine the position of the aperture 2512 relative to the longitudinal axis of the transpedicular access port 2504. In some aspects, the insertion depth and/or insertion angle of the awl 2708 into the bone may be adjusted using an adjustment structure including, for example, the flange 2782, which may be used to deploy and retract the awl 2708. In some aspects, the radius of curvature of the bending of the awl 2708 may be adjusted by changing the dimension and/or shape of the compression cut out(s) 2014 and/or the expansion cut out(s) 2016. In some aspects, the spinal treatment system 2500 may include multiple different awls with compression cut out(s) 2014 and/or expansion cut out(s) 2016 of different dimensions and/or shapes to allow the surgeon to choose between different radius of curvatures of bending of the awl 2708. In some aspects, the bending of the awl 2708 may follow the path of a complex function instead of a partially circular radius of curvature. For example, in some aspects, the path of bending of the awl 2708 may be partially elliptical, parabolic, or hyperbolic. In some aspects, at least the parameters discussed in this paragraph and any other foreseeable parameters may be adjusted alone or in any combination to change to location site of bone void filler delivery. In some aspects, the surgeon(s) and/or user(s) may continuously or periodically monitor the transpedicular access port 2504 and/or the awl 2708 on fluoroscopy to determine the precise position of the transpedicular access port 2504 and/or the awl 2708.

At optional process 4012, the bone tamp 418 may be inserted to into the lumen 2880 of the awl 2708 to clear a cavity proximal to the window 2012. However, it should be understood that in some implementations, instead of or in addition to process 4012, a cavity may be cleared by a stent and/or balloon based on aspects and method discussed anywhere in this disclosure including, for example, disclosure relating to FIGS. 6-8 and/or FIG. 7.

At process 4014, bone void filler 518, 818 may be delivered to the interior of the bone 102. It should be understood that depending on the implementation, bone void filler 518, 818 may be delivered to the interior of the bone 102 based on aspects and method discussed anywhere in this disclosure including, for example, disclosure relating to FIGS. 3-8. In some aspects, as shown in FIGS. 31-32, for example, an injection tube 3102 may be inserted into the lumen 2880 of the awl 2708 in order to deliver bone void filler into the void space created by the awl 2708 and/or the bone tamp 418. In some aspects, the awl 2708 may be completely withdrawn from the working channel 2604 and/or the from the cannula of the guide tube 2506 and an injection tube 3102 may be inserted into the cannula of the guide tube 2506 and the working channel 2604 in order to deliver bone void filler into the void space created by the awl 2708 and/or the bone tamp 418. In some aspects, bone void filler may flow directly through the lumen 2880 of the awl 2708 in order to deliver bone void filler into the void space created by the awl 2708 and/or the bone tamp 418. In some aspects, the awl 2708 may be completely withdrawn from the working channel 2604 and/or the from the cannula of the guide tube 2506 and bone void filler may flow directly through the cannula of the guide tube 2506 and the working channel 2604 in order to deliver bone void filler into the void space created by the awl 2708 and/or the bone tamp 418. It should be appreciated that the awl 2708 may be either consumable or reusable such that, in some aspects, the awl 2708 may be implanted in the patient (i.e., may remain in the patient after surgery) while in other aspects, the awl 2708 may be removed and/or function only as an instrument. In some aspects in which the awl 2708 may remain in the patient after surgery, the inner shaft 2790 may be detached from the distal awl portion 2002 by moving the peg 2042 out of the L-shaped groove 2018 and withdrawing the inner shaft 2790 while the distal awl portion 2002 remains entirely or substantially in the same position.

At optional process 4016, a cap 508 may be coupled to the threads of threaded portion 2620 of the transpedicular access port 2504 to seal the working channel 2604. Thus, it should be appreciated that in some aspects, the threads of the threaded portion 2620 may have the same or substantially the same threading as both the threads of the inner threaded portion 2622 and the threads of the cap 508.

It should be appreciated that any of the processes of method 4000 may be completed in any order. It should also be appreciated that some or all optional steps may be completed depending on the implementation. The order of the steps in method 4000 may be changed indiscriminately as bone void filler 518, 818 is delivered to the interior of the vertebra 102 of a patient.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A vertebral body access port, comprising:
a radiopaque tubular body having a central longitudinal axis and a sidewall extending along said longitudinal axis, the tubular body including a distal portion and a proximal portion interconnected by a central portion having an outer diameter and defining a working lumen having a first diameter;
said distal portion having a distal tip and defining an access window opening extending through said sidewall, the working lumen terminating into a deflection surface formed in the distal portion adjacent the access window, the distal portion further including a radiographic orientation indicator configured to provide radiographic indication of an axial orientation of said access window about said longitudinal axis;
said central portion defining a bone engaging structure on said sidewall to inhibit axial movement of the tubular body along the longitudinal axis, the sidewall being closed along a longitudinal length of said central portion; and
said proximal portion having an enlarged head defining a tool engagement surface for connecting with an insertion tool and an internal passage communicating with said working lumen, said internal passage having a dimension that is equal to or larger than said first diameter, said tool engagement surface having a dimension that is larger than said first diameter, the tool engagement surface further including an alignment feature configured to permit engagement of the insertion tool in a fixed axial orientation relative to the tubular body.

2. The device of claim 1, wherein said radiographic orientation indicator is a notch formed in said distal portion.

3. The device of claim 1, wherein said radiographic orientation indicator is a reduced diameter outer wall adjacent to said access window.

4. The device of claim 1, wherein the bone engaging structure of the central portion includes a helical thread.

5. The device of claim 1, wherein said distal portion further defines a cannula extending along the longitudinal axis configured to receive a guide wire, the cannula having a cannula diameter, the cannula diameter being less than 50% of said first diameter of said working lumen such that the tubular body is guided into position within a bone by the distal portion solely by engagement of the cannula of the distal portion moving along a guidewire.

6. The device of claim 1, wherein in the first diameter of the working lumen is at least 75% of the outer diameter.

7. The device of claim 1, wherein said sidewall has a thickness extending between the outer diameter of the sidewall and the working lumen, said thickness being less than 0.75 mm.

8. The device of claim 1, wherein a thickness of the sidewall is less than 0.75 mm.

9. The device of claim 1, wherein the working lumen has a diameter of at least 5 mm.

10. A transpedicular spinal treatment system, comprising:
an insertion tool comprising an internal passage; and
a vertebral body access port configured to couple to said insertion tool, said vertebral body access port comprising:
a radiopaque tubular body having a central longitudinal axis and a sidewall extending along said longitudinal axis, the tubular body including a distal portion and a proximal portion interconnected by a central portion having an outer diameter and defining a working lumen having a first diameter;
said distal portion having a distal tip and defining an access window opening extending through said sidewall, the working lumen terminating into a deflection surface formed in the distal portion adjacent the access window, the distal portion further including a radiographic orientation indicator configured to provide radiographic indication of an axial orientation of said access window about said longitudinal axis;
said central portion defining a bone engaging structure on said sidewall to inhibit axial movement of the tubular body along the longitudinal axis, the sidewall being closed along a longitudinal length of said central portion; and
said proximal portion having an enlarged head defining a tool engagement surface for connecting with said insertion tool and said internal passage communicating with said working lumen, said internal passage having a dimension that is equal to or larger than said first diameter, said tool engagement surface having a dimension that is larger than said first diameter, the tool engagement surface further including an alignment feature configured to permit engagement of the insertion tool in a fixed axial orientation relative to the tubular body.

11. The system of claim 10, wherein said radiographic orientation indicator is a notch formed in said distal portion.

12. The system of claim 10, wherein said radiographic orientation indicator is a reduced diameter outer wall adjacent to said access window.

13. The system of claim 10, wherein the bone engaging structure of the central portion includes a helical thread.

14. The system of claim 10, wherein said distal portion further defines a cannula extending along the longitudinal axis configured to receive a guide wire, the cannula having a cannula diameter, the cannula diameter being less than 50% of said first diameter of said working lumen such that the tubular body is guided into position within a bone by the distal portion solely by engagement of the cannula of the distal portion moving along a guidewire.

15. The system of claim 10, wherein in the first diameter of the working lumen is at least 75% of the outer diameter.

16. A method comprising:
inserting a vertebral body access port at an insertion location on a bone, said vertebral body access port comprising:
a radiopaque tubular body having a central longitudinal axis and a sidewall extending along said longitudinal axis, the tubular body including a distal portion and a proximal portion interconnected by a central portion having an outer diameter and defining a working lumen having a first diameter;
said distal portion having a distal tip and defining an access window opening extending through said sidewall, the working lumen terminating into a deflection surface formed in the distal portion adjacent the access window, the distal portion further including a radiographic orientation indicator configured to provide radiographic indication of an axial orientation of said access window about said longitudinal axis;
said central portion defining a bone engaging structure on said sidewall to inhibit axial movement of the tubular body along the longitudinal axis, the sidewall being closed along a longitudinal length of said central portion; and
said proximal portion having an enlarged head defining a tool engagement surface for connecting with an insertion tool and an internal passage communicating with said working lumen, said internal passage having a dimension that is equal to or larger than said first diameter, said tool engagement surface having a dimension that is larger than said first diameter, the tool engagement surface further including an alignment feature configured to permit engagement of the insertion tool in a predefined axial orientation relative to the tubular body
orienting said access window to face in a fixed direction based on an orientation of said radiographic orientation indicator;
inserting an awl into the working lumen from a proximal opening of said vertebral body access port; and
moving said awl into said deflection surface and at least partially out of said working lumen through said access window.

17. The method of claim 16, wherein said radiographic orientation indicator is a notch formed in said distal portion.

18. The method of claim 16, wherein said radiographic orientation indicator is a reduced diameter outer wall adjacent to said access window.

19. The method of claim 16, wherein the bone engaging structure of the central portion includes a helical thread.

20. The method of claim 16, wherein said distal portion further defines a cannula extending along the longitudinal axis configured to receive a guide wire, the cannula having a cannula diameter, the cannula diameter being less than 50% of said first diameter of said working lumen such that the tubular body is guided into position within a bone by the distal portion solely by engagement of the cannula of the distal portion moving along a guidewire.
